(19) United States
(12) Patent Application Publication
(10) Pub. No.: US 2025/0034649 A1
(43) Pub. Date: Jan. 30, 2025

(54) METHODS OF ASSESSING SMOLDERING MULTIPLE MYELOMA

(71) Applicant: TELO GENOMICS HOLDINGS CORP., Toronto (CA)

(72) Inventors: Sabine Mai, Winnipeg (CA); Sherif Louis, Toronto (CA); Hans Knecht, Magog (CA)

(21) Appl. No.: 18/710,540

(22) PCT Filed: Nov. 16, 2022
(86) PCT No.: PCT/CA2022/051694

§ 371 Date: May 15, 2024

(30) Foreign Application Priority Data
Nov. 16, 2021 (CA) 3139296

(51) Int. Cl. C12Q 1/6886; C12Q 1/6841

(52) U.S. Cl. CPC C12Q 1/6886; C12Q 1/6841; C12Q 2600/118

(57) ABSTRACT

Provided are methods for prognosing a clinical outcome in a subject or diagnosing the subject with high-risk or stable smoldering multiple myeloma (SMM), comprising determining a 3D telomeres organization signature of a test sample from the subject, the test sample comprising plasma cells, applying a classification model to the 3D telomeres organization signature to obtain an output classification that is indicative of the clinical outcome or diagnosis of the subject. Also provided are methods for treating a subject with high-risk or stable SMM.

### RELATED APPLICATIONS

The present application claims the benefit of priority of Canadian Patent Application no. 3,139,296 filed on Nov. 16, 2021, the contents of which are incorporated herein by reference in their entirety.

### FIELD

The present disclosure relates to methods of differentiating patients with smoldering multiple myeloma and more particularly to methods of differentiating patients with different risk profiles of smoldering multiple myeloma or differentiating patients from other related diseases based on three dimensional telomeric organization.

### BACKGROUND

Multiple myeloma (MM) is a B-cell malignancy characterized by extensive proliferation of plasma cells (PCs) in the bone marrow (BM) and abnormal increase of monoclonal immunoglobulins or M proteins [1]. This aberrant plasma cell proliferation leads to lytic bone lesions, hypercalcemia, kidney failure and severe anemia [1].

MM is the end stage of monoclonal gammopathy of undetermined significance (MGUS) and smoldering multiple myeloma (SMM) [2,3]. All cases of MM are preceded by MGUS or SMM [2,3]. MGUS patients have detectable monoclonal immunoglobulin in blood or urine (<3 g/dL) and clonal bone marrow plasma cells under 10%, but no evidence of end-organ damage or other symptoms. The risk of progression of MGUS to MM is about 1%-2% per year [4,5]. In SMM patients, the levels of M protein in the serum are higher than in MGUS (3 g/dL) and clonal bone marrow plasma cells greater than 10% but less than 60%, with patients still showing no symptoms and no laboratory signs of end-organ damage, but the risk of progression to MM is about 10% per year in the first 5 years, particularly in subjects with blood disorders family history, and/or genetic ethnic background [4, 5, 38].

Despite the development of novel therapeutic strategies and a better understanding of MM biology, this disease remains incurable with a heterogeneous clinical course, where overall survival can range from a few months to over 10 years [6]. Therefore, the development of early reliable surrogate end-points for survival is necessary for better risk stratification, treatment individualization, and future incorporation of novel agents and combinations into the MM management. Among the many prognostic factors and several different risk stratification systems described for MM, the International Staging System (ISS), initially based on serum levels of both β2-microglobulin and albumin [7], has recently incorporated cytogenetic abnormalities and lactate dehydrogenase levels [7]. However, gene expression profile, plasma cell proliferative rate, extramedullary disease, initial presentation as plasma cell leukemia, age, performance status, PET-CT presentation, and comorbidities are also taken into consideration in the clinical perspective on treatment goals and management of MM patients [7].

The relatively modest rate of progression of MGUS to the full stage MM does not imply significant concern for managing MGUS patients. However, the progression of SMM patients to full stage MM has been a burden in the clinic for the past few decades, especially due to the absence of biomarkers that can predict the risk of the individual SMM patient to transition to full stage MM with high specificity [39 & 40].

Furthermore, several clinical studies have shown significant advantage in time-to-progression and overall survival of SMM patients if treated with chemotherapy [41]. Nevertheless, with approximately 90% of the SMM patients presenting a stable form of the SMM disease, the decision to treat all SMM patients with chemotherapy is far from being adopted in standard clinical practice, given the significant side effects associated with these therapies.

In recent years, several approaches have attempted to stratify SMM patients into risk groups including multiple risk stratification models based on traditional clinical risk factors. These models attempted to classify patients into a group with over 50% chance, and a group with under 50% chance, of transitioning to full stage MM within 2 years; however, satisfactory sensitivity was not achieved [39 & 40]. To date, identifying high risk SMM patients that could benefit from chemotherapy or other interventions and confirming the disease stability in low risk SMM patients remain critical clinical unmet needs in the management of MM [39, 40 & 41].

A prominent feature observed in MM cells is a dynamic genomic instability and complexity, which increases with subsequent acquisition of additional genetic abnormalities [8]. The cell-to-cell heterogeneity and presence of multiple subclones affect both prognostic stratification and therapeutic approaches [9]. This intraclonal diversity, where different clones are present at diagnosis and during disease evolution, promotes survival advantage of individual clones upon treatment, selection of minor pre-existing or novel clones, and disease progression [9-12].

Methods for risk stratification or differentiating high risk SMM from low risk SMM are desirable.

### SUMMARY

The present inventors produced diagnostic and prognostic classification models for subjects having smoldering multiple myeloma (SMM) based on 3D telomere analysis of bone marrow samples. For example, it is demonstrated that SMM patients can be classified as having stable SMM not likely to progress to multiple myeloma (MM) within 5 years or as having high-risk SMM likely to progress to MM within 2 years based on their telomere signature.

Accordingly in an aspect, the disclosure includes a method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model comprising one or more of the at least one telomere parameter; and
providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years.

In one embodiment, test sample is a bone marrow sample or a blood sample, optionally a diagnostic bone marrow biopsy sample or a peripheral liquid biopsy blood sample.

In one embodiment, the prognosis or the diagnosis is provided to the subject or the subject's medical professional at time of SMM diagnosis.

In one embodiment, the assaying comprising:
labelling nuclei of the plurality of the plasma cells with a fluorescent nuclear stain or probe, optionally wherein the fluorescent nuclear stain is 4′,6-diamidino-2-phenylindole (DAPI);
tagging telomeres in the plurality of plasma cells through in situ hybridization with a telomere-specific labelled probe,
mounting the test sample using an antifade mounting medium;
3D imaging the test sample; and
measuring on the 3D images values for the at least one telomere parameter to obtain the 3D telomere organization sample signature.

In one embodiment, the telomere-specific labelled probe is a peptide nucleic acid (PNA) probe.

In one embodiment, assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

In one embodiment, the 3D imaging comprises acquiring an image dataset of different planes of 3D q-FISH fluorescent signals and reconstructing a 3D image of the telomeres using deconvolution of the images performed with a constrained iterative algorithm, optionally using fluorescence microscopy.

In one embodiment, the 3D imaging comprises obtaining a stack of at least 50 images with a sample distance of 200 nm along a z direction and 102 nm in each of a x and a y direction.

In one embodiment, the 3D telomere organization sample signature is determined from interphase plasma cells.

In one embodiment, the one or more of and/or the at least telomere parameter comprises an absolute value, a mean, a median, a ratio, a percentile, a quartile, a rank, a range, or a combination thereof.

In one embodiment, the sample is a diagnostic sample.

In one embodiment, the one or more of the at least one telomere parameter of the classification model is selected to have an accuracy of at least 60%, at least 70%, at least 75%, at least 80%, at least 85%, at least 90%, at least 95%, at least 96%, at least 97%, least 98%, at least 99% or 100% in distinguishing between stable SMM and high-risk SMM.

In one embodiment, the classification model comprises at least two, at least three, or at least four of the at least one telomere parameter.

In one embodiment, the one or more of the at least one telomere parameter of the classification model consists of the telomere parameters: nuclear telomere distribution, a/c ratio, and total telomere length.

In one embodiment, the one or more of the at least one telomere parameter of the classification model consists of telomere parameters: a/c ratio and telomere numbers.

In one embodiment, the one or more of the at least one telomere parameter of the classification model consists of the telomere parameters: a/c ratio and nuclear telomere distribution.

In one embodiment, the one or more of the at least one telomere parameter of the classification model consists of the telomere parameter: a/c ratio.

In one embodiment, the one or more of the at least one telomere parameter of the classification model consists of the telomere parameters: a/c ratio and telomere aggregates.

In one embodiment, the subject is a human subject.

Another aspect of the present disclosure is any method described herein, wherein when the subject is prognosed or diagnosed as having an increased likelihood of high-risk SMM, the subject is subsequently treated with one or more of lenalidomide, dexamethasone, siltuximab, daratumumab, bortezomib, elotuzumab, carfilzomib, thalidomide, cyclophosphamide and combinations thereof.

In one embodiment, the subject prognosed or diagnosed as having an increased likelihood of high-risk SMM is subsequently treated with bortezomib and dexamethasone.

In one embodiment, the subject prognosed or diagnosed as having an increased likelihood of high-risk SMM is subsequently treated with siltuximab.

In one embodiment, the subject prognosed or diagnosed as having an increased likelihood of high-risk SMM is subsequently treated with daratumumab, lenalidomide, bortezomib and dexamethasone.

In one embodiment, the subject prognosed or diagnosed as having an increased likelihood of high-risk SMM is subsequently treated with elotuzumab.

In one embodiment, the subject prognosed or diagnosed as having an increased likelihood of high-risk SMM is subsequently treated with carfilzomib, lenalidomide, daratumumab, and dexamethasone, optionally for 3 to 4 cycles as induction therapy.

In one embodiment, the subject prognosed or diagnosed as having an increased likelihood of high-risk SMM is subsequently treated with bortezomib, lenalidomide and dexamethasone.

In one embodiment, the subject prognosed or diagnosed as having an increased likelihood of high-risk SMM is subsequently treated with bortezomib, thalidomide and dexamethasone.

In one embodiment, the subject prognosed or diagnosed as having an increased likelihood of high-risk SMM is subsequently treated with bortezomib, cyclophosphamide and dexamethasone.

In one embodiment, the subject prognosed or diagnosed as having an increased likelihood of high-risk SMM is subsequently treated with lenalidomide.

In one embodiment, the subject prognosed or diagnosed as having an increased likelihood of high-risk SMM is subsequently treated with lenalidomide and dexamethasone.

In one embodiment is any method described herein, wherein when the subject is prognosed or diagnosed as having an increased likelihood of stable SMM or as having an increased likelihood of high-risk SMM, the subject is subsequently monitored.

Another aspect of the present disclosure is A method of monitoring a subject prognosed or diagnosed as having an increased likelihood of stable SMM or as having an increased likelihood of high-risk SMM, comprising:
obtaining a subsequent sample from the subject, the subsequent sample comprising a plurality of plasma cells;
assaying the plurality of plasma cells according to any assaying step described herein, to obtain a 3D telomere organization monitoring signature;
comparing the 3D telomere organization monitoring signature to a 3D telomere organization signature obtained from a previous sample from the subject; and
providing an updated clinical outcome prognosis or an updated diagnosis to the subject having an increased likelihood of stable SMM, wherein the updated clinical outcome prognosis or the updated diagnosis is of no change, an increased likelihood of stable SMM compared to the previous sample, or an increased likelihood of high-risk SMM, or providing an updated clinical outcome prognosis, an updated diagnosis, or an evaluation of treatment response to the subject having an increased likelihood of high-risk SMM, wherein the updated prognosis or diagnosis or the evaluation is an amelioration, stabilization, or worsening of SMM or a symptom thereof.

In an embodiment, the method first comprises obtaining the test sample from the patient.

In an embodiment is a method of treating a subject with smoldering multiple myeloma (SMM) based on a 3D telomere organization signature from the subject, comprising administering to the subject a treatment selected from any treatment described herein when the subject has an increased likelihood of high-risk SMM, or monitoring the subject when the subject has an increased likelihood of stable SMM.

In an embodiment is a method of providing a personalized treatment plan for a subject with smoldering multiple myeloma (SMM) based on a 3D telomere organization signature of the subject, comprising providing the subject the personalized treatment plan to be administered to the subject when the subject has an increased likelihood of high-risk SMM or monitoring the subject when the subject has an increased likelihood of stable SMM, wherein the treatment plan comprises a treatment selected from any treatment described herein.

In an embodiment, the subject having an increased likelihood of high-risk SMM or the subject having an increased likelihood of stable SMM is prognosed or diagnosed as having an increased likelihood of high-risk SMM or an increased likelihood of stable SMM according to any method described herein.

Another aspect of the present disclosure is use of any method described herein for treating a subject with SMM.

In an embodiment is the prognosis or diagnosis determined using any method described herein for use in treating a subject with SMM.

In an embodiment, treating comprises administering to the subject a treatment selected from any treatment described herein when the subject has an increased likelihood of high-risk SMM, or monitoring the subject when the subject has an increased likelihood of stable SMM.

Another aspect of the present disclosure is an assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution; applying a classification model to the 3D telomere organization sample signature to obtain an output classification of stable SMM or high-risk SMM, the model being a model comprising one or more of the at least one telomere parameter; providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years; and selecting a therapy according to any one of claims 20 to 30 for the subject when the subject is identified as having an increased likelihood of high-risk SMM, or monitoring the subject when the subject is identified as having an increased likelihood of stable SMM.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1 is a panel of representative FISH images showing myeloma plasma cells from 3 smoldering myeloma patients. The figure shows the positive staining of CD56 and CD138 specific for myeloma plasma cells in addition to the telomeres labeled using telomeres probe with Cy3 fluorochrome.

FIG. 2 is a series of 3D images showing differences in the 3D nuclear telomere architecture between MGUS, SMM, and MM.

FIG. 3. is a Kaplan-Meier survival analysis graph performed in patients with MGUS, SMM, and MM, using the log-rank test. “dx” refers to diagnosis; “nmtdeath” refers to months to dying censored; and points designated by the symbol “+” refer to cases followed up to that point but did not die. Adjustment for multiple comparisons is shown in Table 4.

FIG. 4. is a Kaplan-Meier survival analysis performed in patients (MGUS, SMM, and MM) cohort using the log-rank test. “nmtdeath” refers to months to dying censored, and points designated by the symbol “+” refer to cases followed up to that point but did not die. Adjustment for multiple comparisons is shown in Table 5.

FIG. 5 is a receiver operating characteristic (ROC) curve showing the ability of a classification model for predicting non-progressing vs progressing SMM. Area under the curve (AUC) is 0.8002. The points within the graph are labelled by observation number.

### DETAILED DESCRIPTION OF THE DISCLOSURE

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

### Definitions

The term “monoclonal gammopathy of undetermined significance” or MGUS is a precursor to multiple myeloma (MM) and is defined as a disease having i) serum monoclonal protein under 3 g/dL, ii) clonal bone marrow plasma cells under 10%, and iii) no end-organ lesions (including hypercalcemia, renal insufficiency, anemia, and bone lesions).

The term “smoldering multiple myeloma” or SMM is asymptomatic precursor to multiple myeloma (MM) and is defined as a disease having i) serum monoclonal protein greater or equal to 3 g/dL, ii) clonal bone marrow plasma cells greater than 10% but less than 60% (however, if plasma cell infiltration is over 10% then 2 g/dL monoclonal protein is considered SMM not MGUS), and iii) no end-organ lesions (including hypercalcemia, renal insufficiency, anemia, and bone lesions). The term “stable SMM” or “SMM-stable” as used herein means SMM that is likely to remain stable for over 5 years (in other words not likely to progress to full stage MM for over 5 years) from time of diagnosis. The term “high-risk SMM” or “SMM-progression” as used herein means SMM that is likely to progress to full stage MM within 1 to 3 years from time of diagnosis, for example within 2 years from time of diagnosis. The assessment of “stable SMM” and “high-risk SMM” is performed based on telomeric organization of plasma cells from a subject sample. As shown herein, high-risk SMM patients can, for example, have increased telomere numbers, total and average telomere length (as demonstrated by their proxies—total and average telomere intensity), telomere aggregates, a/c ratio and/or nuclear volume, and/or changes in nuclear telomere distribution compared to stable SMM patients.

The term “multiple myeloma” or MM or “full stage MM” as used herein means a tumor or cancer composed of cells derived from the hematopoietic tissues of the bone marrow. Multiple myeloma is also known as plasma cell myeloma and is defined by the presence of serum monoclonal protein levels greater than or equal to 20 g/dL, clonal bone marrow plasma cells greater than or equal to 60%, and end organ damage (e.g., lytic bone lesions, anemia, hypercalcemia, renal failure). However, presence of end organ damage is sufficient to diagnose a patient with full stage MM regardless of monoclonal protein levels or clonal bone marrow plasma cell ratio.

The term “three-dimensional (3D) analysis” or “3D analysis” as used herein refers to any technique that allows the 3D visualization of cells, for example involving high resolution deconvolution microscopy, image restoration or deconvolution, visualization and image analysis. An example of 3D image analysis is provided in U.S. Pat. No. 7,801,682, issued Sep. 21, 2010 titled Method of Monitoring Genomic Instability Using 3D Microscopy and Analysis, herein incorporated by reference in its entirety.

The term “telomere numbers” as used herein means a sum of all telomeres and all telomere aggregates identified within a cell and/or group of cells in a test sample, for example a cell or sample from a subject.

The term “telomere aggregates” means telomeres found in close proximity that cannot be further resolved at fluorescence microscopy maximum optical resolution of 200 nm (Vermolen et al., 2005; Mai and Garini, 2006; Mai, 2010). Telomeric aggregates are not typically observed in normal cells.

The term “total telomere length” as used herein means a sum of the length of all telomeres within a given cell or group of cells in a test sample, for example a cell or sample from a subject. The term “average telomere length” as used herein means a mean telomere length, the mean calculated from all telomeres within a given cell or group of cells in a test sample, for example a cell or sample from a subject. Total telomere length and average telomere length are calculated from the relative fluorescent telomeric signal intensity of labelled telomeres, and thus “total telomere intensity” is a proxy for total telomere length and “average telomere intensity” is a proxy for average telomere length. Given that telomeres are regions of repetitive nucleotide sequences, the number of fluorescent probes targeting the repeat sequences that bind to a telomere is a function of its length. Consequently, the intensity of the resulting fluorescent signal is also a function of telomere length. The relative fluorescent telomeric signal intensity represents the length of the telomeres in fluorescent arbitrary units (f.a.u.), which can be subsequently converted into kilo base (kB) pair lengths. This conversion can be calculated through for example a conversion factor derived from cells such as Raji cells, a Burkitt's lymphoma cell line used as a control, as described in Mathur et al., 2014 [42], herein incorporated by reference in its entirety. Any cell line with a defined telomere length can be used in the determination of a conversion factor. As an example, once Raji cells (or other control cell) are harvested, half are used for telomere restriction fragment (TRF) analysis via Southern blot while the rest underwent PNA Q-FISH using identical conditions as set for plasma cells (e.g., using a 63×/1.4 oil plan apochromatic objective lens). Using TeloView™, an average telomere intensity is calculated for the Raji cells. This can be correlated with the telomere length resulting from the TRF analysis, deriving the conversion factor. The intensities can then be converted into base pair lengths using this conversion factor.

The term “a/c ratio” refers to a spatial distribution of telomeres within the nucleus that changes according to the phase of the cell cycle. This cell cycle-dependent distribution of telomeres can be described as an ellipsoid having two axes of equal or similar length (termed “a” and “b”), and one axis of a different length (termed “c”). The ratio between the length of the a-axis and c-axis describes the degree to which this distribution deviates from a sphere, i.e., where a/c=1 (Vermolen et al., 2005). The orientation of the telomere ellipsoid within a sample is not necessarily parallel or perpendicular to the microscope viewing/imaging plane (described by the x- and y-axis), especially in those cases where cells are analyzed within tissue sections.

The term “nuclear telomere distribution” or “three-dimensional (3D) distribution of telomeres in the nuclear space,” also called “3D distribution of telomeres in the nuclear space,” “nuclear telomere distribution”, “distribution of telomeres”, “distance from nuclear centre” or “distance from the nuclear centre and periphery,” in the art, refers to a telomere spatial distribution parameter distinct from a/c ratio that describes the 3D positioning of each telomere within the nuclear volume. 3D distribution of telomeres in the nuclear space can be an absolute value or a relative value. For example, the centre of a nucleus is identified by its x-, y-, and z-axis coordinates as oriented according to the microscope viewing/imaging plane. Each telomere or aggregate also identified by its x-, y-, and z-axis coordinates. For any one telomere, the distance is measured between the nuclear centre, and the telomere to obtain the absolute distance of the telomere from the nuclear center. The 3D vector created by measuring the distance between these two points can be projected past the telomere's coordinates to the periphery of the nucleus in order to obtain the total distance between the nuclear centre and nuclear periphery for that telomere. A ratio can be calculated for each telomere by dividing the distance between the telomere and the nuclear centre by the distance between the nuclear centre and the nuclear periphery. This number can be multiplied by 100 for reporting as a “percent distance from the nucleus”.

The term “nuclear volume” as used herein means the volume of a cell nucleus. The nuclear volume can be calculated for example according to the 3D nuclear 4′,6-diamidino-2-phenylindole staining (DAPI) protocol described in Vermolen B J et al., (2005).

The term “telomeric organization”, “telomere organization” or “telomeres organization” as used herein refers to the 3D arrangement of the telomeres during any phase of a cell cycle and includes such parameters as 3D distribution of telomeres in the nuclear space, number of telomere aggregates per cell, number of telomeres per cell, telomere lengths (or intensities), a/c ratios, and/or nuclear volumes. “Telomere organization” also refers to the size and shape of the telomeric disk, captured for example in an a/c ratio and which is the organized structure formed when the telomeres condense and align during the late G2 phase of the cell cycle.

The term “telomere organization signature” as used herein refers to a specific telomeric signature that classifies the cell for example as stable SMM, high-risk SMM, MGUS or MM. The criteria that define the differences include at least one or a combination of more than one of the following telomere organization parameters 1) 3D distribution of telomeres in the nuclear space, 2) the presence/absence of telomere aggregate(s), 3) telomere numbers per cell, and 4) telomere sizes, 5) a/c ratios and 6) nuclear volumes of a cell or average or median of a group of cells for example at least 5 cells, a least 10 cells, at least 15 cells, at least 20 cells, at least 25 cells or at least 30 cells of a cell sample. The at least one or one or more telomere parameters can be determined by dividing into quartiles telomere values obtained from each of the plurality of cells.

The term “3D telomere organization sample signature” as used herein refers to a telomere organization signature obtained from a cell or group of cells in a test sample, for example a cell or sample from a subject that is suspected of having stable SMM, high-risk SMM, MGUS or MM.

The term “3D telomere organization reference signature” as used herein refers to a telomere organization signature corresponding to or derived from a group of samples and associated with a control population, disease population or disease severity and comprises values for a plurality of telomere organization parameters. For example, a reference telomere organization signature is optionally obtained from a plasma cell sample from a subject or group of subjects that is known as not having SMM, MGUS or MM, as having stable or high-risk SMM, as having MGUS or as having MM.

The term “3D telomere organization monitoring signature” as used herein refers to a telomere organization signature obtained from a cell or a group of cells in a monitoring sample, for example a cell or sample from a subject being monitored for an increased likelihood of having stable SMM.

The term “differences in telomeric organization between for example the stable SMM 3D telomere organization reference signatures and the high-risk SMM 3D telomere organization reference signatures” can be determined, for example, by counting the number of telomeres in the cell, counting the number of telomere aggregates in the cell, measuring the size or volume of any telomere or telomere aggregate, the volume of the nucleus, the nuclear telomere distribution, or the alignment (a/c ratio) of the telomeres, and comparing the counts or measured values, or descriptive statistics derived therefrom, between the cells providing the basis for the stable SMM 3D telomere organization reference signatures and the cells providing the basis for the high-risk SMM 3D telomere organization reference signatures. Once identified, any differences in telomeric organization between the stable SMM 3D telomere organization reference signatures and the high-risk SMM 3D telomere organization reference signatures can, for example, be used to develop a classification model.

The term “percentile” or “percentile telomere value” as used herein refers to a descriptive statistic of the data distribution for a telomere value. A percentile is a value below which a certain percentage of the data falls. For example, the 89^{th} percentile is the value below which 89% of the data is located and above which 11% of the data is located. A percentile telomere value can be used to set a threshold value for subject diagnosis or prognosis. A percentile telomere parameter value can also be used as a telomere parameter within a classification model, for example an algebraic classification model, that discriminates between, for example, stable SMM and high-risk SMM.

The term “quartile” or “quartile telomere value” refers to a descriptive statistic of the data distribution for a telomere value that divides the total data into 4 parts. The first quartile (also called Q1) is the 25^{th} percentile, below which 25% of the data is present; the second quartile (also called Q2, or the median) is the 50^{th} percentile, below which 50% of data is present; the third quartile (also called Q3) is the 75^{th} percentile, below which 75% of the data is present; and the fourth quartile (also called Q4) is the 100^{th} percentile, below which 100% of the data is present. A quartile telomere value can be used to set a threshold value for subject diagnosis or prognosis. A quartile telomere parameter value can also be used as a telomere parameter within a classification model, for example an algebraic classification model, that discriminates between, for example, stable SMM and high-risk SMM.

The term “classification model” refers to a model that uses input, for example one or a plurality of values corresponding to at least one or one or more telomere parameters obtained from assaying a diagnostic sample that were found to be significantly different between patient groups and can classify the input into output categories, for example binary categories such as stable SMM and high-risk SMM. A classification model can, for example, be an algebraic classification model. The associated terms “to classify”, “classifying”, and “performing classification” refer to the operations performed by a classification model.

The term “sample” as used herein refers to any biological fluid (e.g., bone marrow or blood) comprising a cell (e.g., plasma lineage cell) from a subject including a sample from a test subject, i.e. a test sample, such as from a subject, for example, with SMM, wherein the test sample comprises abnormal and/or cancer cells, and a control sample from a control subject, e.g., a subject without SMM. For example, the sample comprises a bone marrow sample or a liquid biopsy blood sample comprising plasma cells. The sample can comprise a liquid biopsy blood sample, for example a peripheral liquid biopsy blood sample, a fractionated liquid biopsy blood sample, a bone marrow sample, a biopsy, a frozen sample, a fresh sample, a cell sample, and/or a paraffin embedded sample or section thereof. For example, the sample is a diagnostic bone marrow biopsy sample.

As used herein, the term “cell” includes more than one cell or a plurality of cells or portions of cells. The sample may be from any animal, in particular from humans, and may be biological fluids (such as blood, serum, or bone marrow), tissue, or organ. The term “test cell” is for example a cell obtained or derived from a subject with SMM. In such an embodiment, the test cell includes, but is not limited to, a hematopoietic cancer cell or a cancer precursor cell. The term “control cell” is a suitable comparator cell e.g. a cell that is known of not having a hematopoietic cancer (e.g. negative control) or that is known as having a hematopoietic cancer or precursor syndrome (e.g. positive control).

The term “control” as used herein refers to a suitable comparator subject, sample, cell or cells such as non-SMM subject, or liquid biopsy blood sample, cell or cells from such a subject, for comparison to a subject, sample (e.g. test sample) cell or cells from a subject having SMM; or an untreated subject, cell or cells, for comparison to a treated subject, cell or cells, according to the context. “Control” can also refer to a value or reference signature representative of a control subject, cell and/or cells and/or a population of subjects etc.

The term “prognosis” as used herein refers to an expected course of clinical disease. The prognosis provides an indication of disease progression and includes for example, an indication of likelihood of recurrence, progression to cancer, e.g. MM, metastasis, death due to disease, tumor subtype or tumor type. The prognosis can comprise a good prognosis which corresponds to a good clinical outcome relative to the spectrum of possible clinical outcomes for the specific cancer or syndrome, and a poor prognosis, which corresponds to a poor clinical outcome relative to the spectrum of possible clinical outcomes for the specific cancer or syndrome. As used herein, “good prognosis” means a probable course of disease or disease outcome that has reduced morbidity and/or reduced mortality compared to the average for the disease or condition. As used herein, “poor prognosis” means a probable course of disease or disease outcome that has increased morbidity and/or increased mortality compared to the average for the disease or condition.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. For example, a subject identified as high-risk SMM (e.g. having a high-risk of progressing to MM) can be treated to prevent or delay progression to MM. Treatment methods comprise administering to a subject a therapeutically effective amount of a suitable therapeutic compound including for example therapeutic compounds described herein and optionally consists of a single administration, or alternatively comprises a series of applications. For example, the compounds described herein may be administered at least once a week. However, in another embodiment, the compounds may be administered to the subject from about one time per three weeks, or about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. In some instances, chronic administration may be required. For example, the compounds are administered to the subject in an amount and for a duration sufficient to treat the patient. “Treating” and “Treatment” can also mean monitoring progression of a subject without administering an amount of a compound such as one of the compounds disclosed herein. For example, where the subject is identified as having stable SMM (e.g. having low risk of progression to MM)_the treatment can comprise actively monitoring or surveying the subject, for example by routinely (e.g. once a year or every other year) assessing one or more 3D telomere organization parameters in plasma cells of the subject. This allows the subject to receive beneficial care without having to receive unnecessary therapeutic compound or other such treatment.

As used herein, the phrase “effective amount” or “therapeutically effective amount” means an amount effective, at dosages and for periods of time necessary to achieve a desired result or predicted to achieve a desired result.

The term “subject” also referred to as “patient”, as used herein includes all members of the animal kingdom including mammals, and suitably refers to humans.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 25%, 0.1-20%, or 1-25%, 1-20%, 1%-15%, preferably 10%, most preferably about 5% of the number to which reference is being made.

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods

The present disclosure provides in various embodiments, methods for assessing and stratifying SMM patients as likely to remain stable or as high-risk and likely to progress to MM. The methods provided can also be used to provide appropriate treatments to SMM patients more likely to progress to MM.

Technology for three-dimensional (3D) organization of telomeres in cells such as abnormal or cancer cells has been developed and is disclosed in U.S. Pat. No. 7,801,682, issued Sep. 21, 2010, and U.S. Pat. No. 9,963,745, issued May 8, 2018, each of which are incorporated herein by reference in their entirety.

The inventors' research has shown that the 3D organization of telomeres is altered in cancer cells (Louis et al., 2005; Chuang et al., 2004; Mai and Garini, 2006). This basic finding led to an understanding of genetic changes in early cancer cells and proved that telomere organization is key to genome stability vs. instability (Mai and Garini, 2006; Mai and Garini, 2005; Louis et al., 2005): The inventors have demonstrated that each nucleus has a specific telomeric signature that defines it as normal or aberrant. Six criteria (also called “telomere organization parameters” herein) define this difference; 1) nuclear telomere distribution, 2) the presence/absence of telomere aggregate(s), 3) telomere numbers per cell, and 4) telomere sizes, 5) a/c ratios and 6) nuclear volumes.

As disclosed herein, 3D telomere analysis of diagnostic bone marrow samples is unexpectedly able to provide prognosis and diagnosis information in SMM subjects. For example, it is demonstrated that SMM patients can be classified as having stable SMM or as having high-risk SMM based on their telomere signature. For example, multivariate analysis of the telomere signature of an SMM patient can reliably classify the signature as indicative of stable SMM or high-risk SMM using telomere parameters, for example total telomere length, a/c ratio, nuclear volume, and nuclear telomere distribution.

Accordingly in an aspect, the disclosure includes a method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model comprising one or more of the at least one telomere parameter;
providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years.

The one or more telomere parameter or one or more features thereof of the classification model can be determined by comparing differences and similarities between a stable SMM 3D telomere organization reference signature and a high-risk SMM 3D telomere organization reference signature, the stable SMM 3D telomere organization reference signature defining for one or more of the parameters of a 3D telomere organization signature associated with stable SMM and the high-risk SMM 3D telomere organization reference signature defining for one or more of the parameters a 3D telomere organization signature associated with high-risk SMM. The comparing can be done, for example, by univariate analysis, multivariate analysis, or a combination thereof.

For example, a classification model can include one or more telomere parameter identified as significant in Table 2, for example, total telomere length, average telomere length, telomere numbers, telomere aggregates, a/c ratio or nuclear volume.

For example, the telomere parameters a/c ratio, nuclear volume, nuclear telomere distribution, and/or total telomere length and/or one or more features thereof were identified by univariate analysis as being significantly different between the stable SMM 3D telomere organization reference signatures and the high-risk SMM 3D telomere organization reference signatures. These can, for example, be used to identify the telomere parameters or features thereof for classification models.

“A test sample from a subject having SMM” in any method disclosed herein is, for example, a sample from a subject that meets the clinical criteria for SMM, optionally known or unknown. The sample may for example be a diagnostic sample, wherein the subject and/or sample meet SMM criteria or may be obtained from a subject that has been diagnosed with SMM.

In an embodiment according to any method described herein, the test sample is a bone marrow sample. For example, the bone marrow sample is a diagnostic bone marrow biopsy sample. In another embodiment, the plasma cell is a peripheral liquid blood plasma cell that may be obtained through a blood sample, for example a liquid biopsy of peripheral blood. A plasma cell for example is characterized by abundant basophile cytoplasm, a prominent Golgi zone and an eccentrically located nucleus. In addition, they express the specific marker CD138 for immunohistochemical detection. Plasma cells can readily be identified using a light microscopic technique in bone marrow and blood smears. These cells can be isolated using methods known in the art.

In an embodiment according to any method described herein, the prognosis or the diagnosis is provided to the subject or the subject's medical professional at time of SMM diagnosis.

In an embodiment according to any method described herein, the test sample is a diagnostic biopsy. A diagnostic biopsy is taken for example prior to treatment. A diagnostic sample is a sample of tissue which defines the first time a disease is under investigation. For example, a bone marrow node biopsy can reveal a subject has monoclonal gammopathy of undetermined significance (MGUS), stable SMM, high-risk SMM or MM. The diagnostic biopsy can provide a basis to start a specific treatment.

In an embodiment according to any method described herein, the assaying comprises:
labelling nuclei of the plurality of plasma cells with a fluorescent nuclear stain or probe, for example wherein the fluorescent nuclear stain is 4′,6-diamidino-2-phenylindole (DAPI);
tagging telomeres in the plurality of plasma cells through in situ hybridization with a telomere-specific labelled probe;
mounting the test sample using an antifade mounting medium;
3D imaging the test sample; and
measuring on the 3D images values for the at least one telomere parameter to obtain the 3D telomere organization sample signature.

In an embodiment according to any method described herein, the telomere-specific labelled probe is a Cy3-labelled peptide nucleic acid (PNA) probe. In other embodiments, the labelled probe is a PNA probe linked to another fluorophore, for example Cy2, GFP, or Alexa Fluor 594.

Malignant plasma cells can be identified, distinguished, and/or isolated from other cells in a sample, for example by double-labeling the sample for peptides CD138 and CD56, given that malignant plasma cells can express both peptides CD138 and CD56.

Accordingly, in an embodiment according to any method described herein, the assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

In an embodiment according to any method described herein, the 3D imaging comprises acquiring an image dataset of different planes of 3D q-FISH fluorescent signals and reconstructing a 3D image of the telomeres using deconvolution of the images performed with a constrained iterative algorithm. The 3D imaging may be obtained according to the method described herein. For example, the 3D imaging comprises using fluorescence microscopy using a 63×/1.4 oil plan apochromatic objective lens. In an embodiment, the 3D imaging comprises obtaining a stack of at least 50 images with a sample distance of 200 nm along a z direction and 102 nm in each of a x and a y direction.

In an embodiment according to any method described herein, the 3D telomere organization sample signature is determined from interphase plasma cells.

Each of the one or more telomere parameters of the classification model can, for example, be represented as an absolute value or as a descriptive statistic. Accordingly, in some embodiments according to any method described herein, the one or more telomere parameters of the classification model comprises an absolute value, a mean, a median, a ratio, a percentile, a quartile, a rank, and a range, or a combination thereof. For example, a ratio can be a percentage. In an embodiment, the classification model comprises a rank. In an embodiment, the classification model comprises a quartile. In an embodiment, classification model comprises a range. In an embodiment, the range is an inter-quartile range, for example the range between Q1 and Q2, the range between Q1 and Q3, or the range between Q1 and Q4.

In an embodiment, the telomere parameter is a weighted value.

In an embodiment according to any method described herein, the sample is a diagnostic sample.

Several classification models are disclosed herein that can distinguish between stable SMM and high-risk SMM. These classification models include, for example, one of the following combinations of one or more of the telomere parameters: nuclear telomere distribution, a/c ratio, and total telomere length; a/c ratio and telomere numbers; a/c ratio and nuclear telomere distribution; a/c ratio; and a/c ratio and telomere aggregates.

In an embodiment, the one or more telomere parameters of a classification model are selected to have an accuracy of at least 60%, at least 70%, at least 75%, at least 80%, at least 85%, at least 90%, at least 95%, at least 96%, at least 97%, least 98%, at least 99% or 100% in distinguishing between stable SMM and high-risk SMM. In an embodiment, accuracy is determined by the area under a receiver operating characteristic (ROC) curve. In an embodiment according to any method described herein, the 3D telomere organization sample signature or the classification model comprises at least two telomere parameters. In an embodiment according to any method described herein, the 3D telomere organization sample signature or the classification model comprises at least three telomere parameters. In an embodiment according to any method described herein, the 3D telomere organization sample signature or the classification model comprises at least four telomere parameters.

In an embodiment, the 3D telomere organization sample signature and/or the one or more of the at least one telomere parameter of the classification model consists of the telomere parameters: nuclear telomere distribution, a/c ratio, and total telomere length.

In an embodiment, the 3D telomere organization sample signature and/or the one or more of the at least one telomere parameter of the classification model consists of the telomere parameters: a/c ratio and telomere numbers.

In an embodiment, the 3D telomere organization sample signature and/or the one or more of the at least one telomere parameter of the classification model consists of the telomere parameters: a/c ratio and nuclear telomere distribution.

In an embodiment, the 3D telomere organization sample signature and/or the one or more of the at least one telomere parameter of the classification model consists of the telomere parameter: a/c ratio.

In an embodiment, the 3D telomere organization sample signature and/or the one or more of the at least one telomere parameter of the classification model consists of the telomere parameters: a/c ratio and telomere aggregates.

In an embodiment, the subject is a human subject.

Approximately 10-15% of patients diagnosed with SMM will transition into full stage MM. The current practice for SMM patients is to monitor without treatment for the fact that the high-risk 10-15% of patients cannot be accurately identified. The methods herein described unexpectedly allow for early and accurate identification of subgroups of SMM subjects, namely subjects with high-risk SMM, who may benefit from receiving early treatment to delay or prevent transition to full stage MM.

Subjects identified as high-risk SMM may benefit from treatment modalities similar to those used for MM patients. MM patients can be treated for example with bortezomib, lenalidomide, dexamethasone (VRd). Alternative bortezomib-containing regimens include, for example, bortezomib-thalidomide-dexamethasone (VTd) or bortezomib-cyclophosphamide-dexamethasone (VCd), and can be used instead of VRd.

Accordingly, another aspect disclosed herein relates to a method of treating a subject with high-risk SMM, comprising identifying (e.g., through clinical outcome prognosis and/or diagnosis) the subject as having an increased likelihood of high-risk SMM or stable SMM; and if the subject is identified as likely to have high-risk SMM, administering to the subject a treatment herein disclosed, and if the subject is identified as likely to have stable SMM, monitoring the subject.

In an embodiment, the subject is identified (e.g., through clinical outcome prognosis and/or diagnosis) as having an increased likelihood of high-risk SMM or stable SMM according to any method disclosed herein.

In an embodiment, the subject identified as having an increased likelihood of high-risk SMM, optionally by any method described herein, is subsequently treated with an amount, for example a therapeutically effective amount, of one or more of lenalidomide, dexamethasone, siltuximab, daratumumab, bortezomib, elotuzumab, carfilzomib, thalidomide, cyclophosphamide and combinations thereof, for example wherein the therapeutically effective amount with respect to a combination means an amount of each compound of the combination that is therapeutically effective when combined with the other compounds of the combination.

For example, the treatment is or comprises bortezomib and dexamethasone.

For example, the treatment is or comprises siltuximab.

For example, the treatment is or comprises daratumumab, lenalidomide, bortezomib and dexamethasone.

For example, the treatment is or comprises elotuzumab.

For example, the treatment is or comprises carfilzomib, lenalidomide, daratumumab, and dexamethasone, optionally for 3 to 4 cycles as induction therapy.

For example, the treatment is or comprises bortezomib, lenalidomide and dexamethasone.

For example, the treatment is or comprises bortezomib, thalidomide and dexamethasone.

For example, the treatment is or comprises bortezomib, cyclophosphamide and dexamethasone.

For example, the treatment is or comprises lenalidomide.

For example, the treatment is lenalidomide and dexamethasone.

A subject identified as having an increased likelihood of stable SMM may benefit from monitoring to determine whether the subject has remained in stable SMM or whether the subject has transitioned to increased likelihood of high-risk SMM when they may benefit from treatment modalities for MM patients. A subject identified as having an increased likelihood of high-risk SMM may also benefit from monitoring, for example monitoring the response to any of the treatments disclosed herein.

Accordingly, in an embodiment, the subject prognosed or diagnosed as having an increased likelihood of stable SMM or as having an increased likelihood of high-risk SMM is subsequently monitored.

Another embodiment includes a method of monitoring a subject prognosed or diagnosed as having an increased likelihood of stable SMM, or as having an increased likelihood of high-risk SMM comprising:
obtaining a subsequent sample from the subject, the subsequent sample comprising a plurality of plasma cells;
assaying the plurality of plasma cells according to the assaying step of any method described herein, to obtain a 3D telomere organization monitoring signature;
comparing the 3D telomere organization monitoring signature to a 3D telomere organization signature obtained from a previous sample from the subject; and
providing an updated clinical outcome prognosis or an updated diagnosis to the subject having an increased likelihood of stable SMM, wherein the updated clinical outcome prognosis or the updated diagnosis is of no change, an increased likelihood of stable SMM compared to the previous sample or an increased likelihood of high-risk SMM, or providing an updated clinical outcome prognosis, an updated diagnosis, or an evaluation of treatment response to the subject having an increased likelihood of high-risk SMM, wherein the updated prognosis or diagnosis or the evaluation is an amelioration, stabilization, or worsening of SMM or a symptom thereof.

For example, the subsequent sample can be obtained, for example, 6 months, 1 year, 2 years, 3 years, 4 years, 5 years, or 10 years following prognosis or diagnosis as having an increased likelihood of stable SMM or as having an increased likelihood of high-risk SMM, optionally wherein a new subsequent sample from the subject is obtained, for example, every 6 months, every year or every other year.

In an embodiment, any method described herein first comprises obtaining the test sample from the patient.

Another embodiment herein disclosed is a method of treating a subject with smoldering multiple myeloma (SMM) based on a 3D telomere organization signature from the subject or a classification model, comprising administering to the subject a treatment, for example a treatment selected from the treatments disclosed herein when the subject has an increased likelihood of high-risk SMM, or monitoring the subject when the subject has an increased likelihood of stable SMM.

In an embodiment, the method of treating a subject with smoldering multiple myeloma (SMM) based on a 3D telomere organization signature from the subject or a classification model is performed on a subject that has been prognosed or diagnosed as having an increased likelihood of high-risk SMM or an increased likelihood of stable SMM according to any method provided herein.

Another embodiment herein disclosed is a method of providing a personalized treatment plan for a subject with smoldering multiple myeloma (SMM) based on a 3D telomere organization signature of the subject or a classification model, comprising providing the subject a treatment plan to be administered to the subject when the subject has an increased likelihood of high-risk SMM and monitoring the subject when the subject has an increased likelihood of stable SMM.

In an embodiment, the method of providing a personalized treatment plan for a subject with smoldering multiple myeloma (SMM) based on a 3D telomere organization signature of the subject is performed on a subject that has been prognosed or diagnosed as having an increased likelihood of high-risk SMM or an increased likelihood of stable SMM according to any method provided herein.

Another embodiment herein disclosed is use of a method described herein for treating a subject with smoldering multiple myeloma (SMM), for example, wherein treating comprises administering to the subject a treatment described herein when the subject has an increased likelihood of high-risk SMM, or monitoring the subject when the subject has an increased likelihood of stable SMM.

Yet other embodiments herein disclosed are uses of the prognosis determined using a method described herein in treating a subject with smoldering multiple myeloma (SMM), for example, wherein treating comprises administering to the subject a treatment such as a treatment described herein when the subject has an increased likelihood of high-risk SMM, or monitoring the subject when the subject has an increased likelihood of stable SMM.

Another aspect herein disclosed is an assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution; applying a classification model to the 3D telomere organization sample signature to obtain an output classification of stable SMM or high-risk SMM, the model being for example an algebraic model comprising one or more of the at least one telomere parameters; providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years; and selecting a therapy according any one herein disclosed for the subject when the subject is identified as having an increased likelihood of high-risk SMM, or monitoring the subject when the subject is identified as having an increased likelihood of stable SMM.

In an embodiment according to any method herein disclosed, the classification model or the 3D telomere organization signature consists of one of the following combinations of the telomere parameters: nuclear telomere distribution, a/c ratio, and total telomere length; a/c ratio and telomere numbers; a/c ratio and nuclear telomere distribution; a/c ratio; and a/c ratio and telomere aggregates.

Another embodiment is a method of clinical outcome prognosis or of diagnosis, comprising:
assaying a plurality of plasma cells using three-dimensional (3D) quantitative fluorescence in situ hybridization (q-FISH) to obtain a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution, the plurality of plasma cells previously obtained from a test sample from a subject having smoldering multiple myeloma (SMM), for example wherein the subject has been diagnosed with SMM;
comparing the 3D telomere organization sample signature to a stable SMM 3D telomere organization reference signature and/or a high-risk SMM 3D telomere organization reference signature, the stable SMM 3D telomere organization reference signature defining for one or more of the parameters of a 3D telomere organization signature associated with stable SMM and the high-risk SMM 3D telomere organization reference signature defining for one or more of the parameters a 3D telomere organization signature associated with high-risk SMM;
identifying differences and/or similarities between the 3D telomere organization test sample signature and the stable SMM 3D telomere organization reference signature and/or the high-risk SMM 3D telomere organization reference signature; and
providing the clinical outcome prognosis or the diagnosis to the subject according to the identified differences and/or similarities, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years.

A 3D telomere organization sample signature from a subject with SMM most similar to a high-risk SMM 3D telomere organization reference signature, (e.g. as compared to a stable SMM 3D telomere organization reference signature), for example, indicates the subject has an increased risk to progress to MM within 2 years (e.g. compared to a subject with a 3D telomere organization sample signature most similar to a stable SMM 3D telomeres organization reference signature).

In an embodiment, a subject having SMM identified to have a signature resembling a high-risk SMM 3D telomere organization reference signature is treated for MM.

In an embodiment, wherein the comparing comprises comparing the 3D telomeres organization sample signature with a reference 3D telomeres organization signature comprising the telomere numbers, the total telomere length, the average telomere length, the telomere aggregates, the a/c ratio, the nuclear volume, and/or the nuclear telomere distribution, wherein detecting an increase in the telomere numbers, an increase in the total telomere intensity, an increase in the average telomere intensity, an increase in the telomere aggregates, an increase in the a/c ratio and/or an increase in the nuclear volume and/or a change in nuclear telomere distribution is indicative of likelihood of SMM with high-risk of progression to MM.

The values for the at least one telomere parameter may be represented in the 3D telomere organization sample signature, the stable SMM 3D telomere organization reference signature, or the high-risk SMM 3D telomere organization reference signature as descriptive statistics. Accordingly, in some embodiments, the values for the at least one telomere parameter are represented in the 3D telomere organization sample signature, the stable SMM 3D telomere organization reference signature, or the high-risk SMM 3D telomere organization reference signature as a descriptive statistic selected from a mean, a median, a ratio, a percentile, a quartile, a rank, and a range, or a combination thereof.

As shown in Tables 3 and 7, the inventors have determined two distinct telomeric signatures within a group of SMM patients. These groups are stable SMM and high-risk SMM.

In an embodiment, the 3D telomere organization sample signature or classification model consists of one or more of the telomere parameters: nuclear telomere distribution, a/c ratio, and total telomere length.

In an embodiment, the telomere organization sample signature and/or the one or more of the at least one telomere parameter of the classification model consists of one or more of the telomere parameters: a/c ratio and telomere numbers.

In an embodiment, the telomere organization sample signature and/or the one or more of the at least one telomere parameter of the classification model consists of one or more of the telomere parameters: a/c ratio and nuclear telomere distribution.

In an embodiment, the telomere organization sample signature and/or the one or more of the at least one telomere parameter of the classification model consists of one or more of the telomere parameter: a/c ratio.

In an embodiment, the telomere organization sample signature and/or the one or more of the at least one telomere parameter of the classification model consists of one or more of the telomere parameters: a/c ratio and telomere aggregates.

In an embodiment, the telomere parameter is determined by dividing telomere values into quartiles, as described herein.

For example, the stable SMM 3D telomeres organization reference signature is defined by a median or average number of telomeres per cell equal to or less than 38, equal to or less than 34, equal to or less than 32, equal to or less than 30, equal to or less than 28, equal to or less than 26, equal to or less than 24, equal to or less than 22 or equal to or less than 20.

For example, the high-risk SMM 3D telomeres organization reference signature is defined by a median or average number of telomeres per cell greater than 38, greater than 40, greater than 42, greater than 44, greater than 46, greater than 48 or greater than 50.

For example, the stable SMM 3D telomeres organization reference signature is defined by a median or average number of aggregates per cell equal to or less than 3.5, equal to or less than 3.0, equal to or less than 2.5, equal to or less than 2.0, equal to or less than 1.5 or equal to or less than 1.0.

For example, the high-risk SMM 3D telomeres organization reference signature is defined by a median or average number of aggregates per cell greater than 3.5, greater than 4.0, greater than 4.5, greater than 5.0, greater than 5.5 or greater than 6.0.

For example, the stable SMM 3D telomeres organization reference signature is defined by a median or average a/c ratio per cell equal to or less than 6.5, equal to or less than 6.3, equal to or less than 6.1, equal to or less than 5.9, equal to or less than 5.7, equal to or less than 5.5, equal to or less than 5.3, equal to or less than 5.1 or equal to or less than 4.9.

For example, the high-risk SMM 3D telomeres organization reference signature is defined by a median or average a/c ratio per cell greater than 6.5 greater than 6.7, greater than 6.9, greater than 7.1, greater than 7.3 or greater than 7.5.

For example, the stable SMM 3D telomeres organization reference signature is defined by a median or average, average telomere intensity per cell equal to or less than 16,500, equal to or less than 160,000, equal to or less than 15,500, equal to or less than 15,000, equal to or less than 14,500, equal to or less than 14,000, equal to or less than 13,500 or equal to or less than 13,000 fluorescence arbitrary units (f.a.u.).

For example, the high-risk SMM 3D telomeres organization reference signature is defined by a median or average, average telomere intensity per cell greater than 16,500, greater than 17,000, greater than 17,500, greater than 18,000, greater than 18,500, greater than 19,000, greater than 19,500 or greater than 20,000 f.a.u.

For example, the stable SMM 3D telomeres organization reference signature is defined by a median or average total telomere intensity per cell equal to or less than 600,000, equal to or less than 550,000, equal to or less than 500,000, equal to or less than 450,000 or equal to or less than 400,000 f.a.u.

For example, the high-risk SMM 3D telomeres organization reference signature is defined by a median or average total telomere intensity per cell greater than 600,000, greater than 650,000, greater than 700,000, greater than 750,000, greater than 800,000, greater than 850,000, greater than 900,000, greater than 950,000 or greater than 1,00,000 of f.a.u.

For example, the stable SMM 3D telomeres organization reference signature is defined by a median or average nuclear volume per cell equal to or less than 450,000, equal to or less than 400,000, equal to or less than 350,000, equal to or less than 300,000 or equal to or less than 250,000 μm2.

For example, the high-risk SMM 3D telomeres organization reference signature is defined by a median or average nuclear volume per cell greater than greater than 450,000, greater than 500,000, greater than 550,000, greater than 600,000, greater than 650,000 or greater than 700,000 μm2.

For example, the stable SMM 3D telomeres organization reference signature comprises one or more 3D telomere parameter values as set out in Table 3. For example, the high-risk SMM 3D telomeres organization reference signature comprises one or more 3D telomere parameter values as set out in Table 3.

In an embodiment, the subject is identified as likely to have high-risk SMM or stable SMM according to the method disclosed herein.

In an embodiment, the 3D telomeric organization signature is determined using 3D quantitative fluorescence in situ hybridization (3D q-FISH).

In an embodiment, 3D q-FISH comprises labelling the telomeres with a telomere FISH protocol as described in the Examples. For example, the telomere FISH protocol can be performed by using Cy3-labelled peptide nucleic acid (PNA) probes (DAKO). In an embodiment, telomere labeling is done through in situ hybridization. In an embodiment, the nucleus is labeled with a fluorescent nuclear stain or probe, for example wherein the fluorescent nuclear stain is 4′,6-diamidino-2-phenylindole (DAPI). In an embodiment, 3D q-FISH comprises labelling CD138 in the plurality of plasma cells with a CD138-specific antibody linked to a fluorescent label and/or labelling peptide CD56 in the plurality of plasma cells with a CD56-specific antibody linked to a fluorescent label prior to the mounting of the test sample. In an embodiment, the sample is mounted, optionally using an antifade mounting medium.

For example, 3D Image Acquisition and Analysis can be performed as described in the Examples. For example, the 3D images can be obtained using a 3D imaging system that enables an Abbe resolution of 200 nm, for example an Axiolmager Z1 (Zeiss) microscope using a 63×/1.4 oil plan apochromatic objective lens. In an embodiment, imaging is done on interphase plasma cells. In an embodiment, 3D imaging of telomeres is performed by acquiring a stack of up to 80 images along the z-axis (specimen depth), with a z-axis step of 0.2 μm between each image in the stack. In an embodiment, The sampling distance in both the x- and y-planes is 102 nm. In an embodiment, the exposure time for Cy3 (telomeres) is maintained at 100 milliseconds. In an embodiment, images are acquired in multichannel mode. In an embodiment, the images are deconvolved using a constrained iterative algorithm. In an embodiment, the method uses Teloview™. For example, Teloview™ can be used to determine the 3D telomere organization of a cell. TeloView™ is capable of scanning multiple cells, multiple cell types and multiple patients at one time. In an embodiment, the 3D Image Acquisition can involve analyzing plasma cells with deconvolution module and rendering module. In an embodiment, the 3D image can consist of a stack of at least 51 images with a sampling distance of 200 nm along the z direction and 102 nm in the x and y direction for every fluorochrome.

In an embodiment, the 3D Image Analysis for telomeres involves telomere measurements done with TeloView™. In an embodiment each telomere or telomere aggregate is given a set of coordinates (x, y, z) denoted by crosses on the screen in TeloView™. In an embodiment, total telomere length, average telomere length, telomere numbers, telomere aggregates, a/c ratio, nuclear volume, nuclear telomere distribution, or a combination thereof is measured using TeloView™.

In an embodiment, statistical analysis is performed on measures derived from total telomere length, average telomere length, telomere numbers, telomere aggregates, a/c ratio, nuclear volume, nuclear telomere distribution, or a combination thereof. In an embodiment, statistical analysis is used to identify differences and similarities between a stable SMM 3D telomere organization reference signature and a high-risk SMM 3D telomere organization reference signature, the stable SMM 3D telomere organization reference signature defining for one or more of the parameters of a 3D telomere organization signature associated with stable SMM and the high-risk SMM 3D telomere organization reference signature defining for one or more of the parameters a 3D telomere organization signature associated with high-risk SMM. In an embodiment, bivariate analysis of the stable SMM 3D telomere organization reference signature and the high-risk SMM 3D telomere organization reference signature is used to identify a list of potential telomere parameters or features thereof to use in a classification model, optionally an algebraic model. In another embodiment, statistical analysis comprises multivariate analysis. In an embodiment, multivariate analysis comprises using forward selection methodologies, backward selection methodologies or a combination thereof. In an embodiment, multivariate analysis identifies one or more telomere parameters or features to use in a classification model, optionally an algebraic model. In an embodiment, the accuracy of the classification model is determined by the area under the receiver operating characteristic (ROC) curve. In an embodiment, the one or more telomere parameters or features thereof of the classification model comprises an absolute value, a mean, a median, a ratio, a percentile, a quartile, a rank, and a range, or a combination thereof. For example, a ratio can be a percentage. In an embodiment, the classification model comprises a rank. In an embodiment, the classification model comprises a quartile. In an embodiment, classification model comprises a range. In an embodiment, the range is an inter-quartile range, for example the range between Q1 and Q2, the range between Q1 and Q3, or the range between Q1 and Q4. In an embodiment, the classification model is applied to the 3D telomere organization sample signature of a subject having SMM to obtain an output classification of stable SMM or high-risk SMM.

In an embodiment, the classification model can comprise a rank of the one or more of the at least one telomere parameters. For example, the one or more of the at least one telomere parameters can be ranked from lowest to highest. In another embodiment, the classification model can comprise calculating an inter quartile range for one or more of the at least one telomere parameter, for example the 75^{th} to 25^{th} percentile range. In another embodiment the interquartile range is calculated for a ranked at least one telomere parameter. In one embodiment, the interquartile range can be an aggregated range for two or more of the telomere parameters. In one embodiment, the two or more telomere parameters are a/c ratio, telomere distribution and number of telomeres. In an embodiment, one or more of the at least one telomere values is weighted. The one or more of the at least one telomere parameter (e.g. telomere parameter consists of parameter name and value) can be added in some embodiments. The classification models as described herein can predict the probability that a subject has high risk SMM. A threshold probability can be selected such that if the probability is above the selected threshold, the subject is identified as high risk SMM and if below the selected threshold the subject is identified as stable SMM.

The present disclosure also provides the following embodiments:

Embodiment 1. A method for prognosing a clinical outcome in a subject having smoldering multiple myeloma (SMM), comprising:
assaying a plurality of plasma cells using three-dimensional (3D) quantitative fluorescence in situ hybridization (q-FISH) to obtain a 3D telomeres organization sample signature, the 3D telomeres organization sample signature comprising at least one parameter selected from telomere numbers, total telomere intensity, average telomere intensity, telomere aggregates, a/c ratio and nuclear volume, the plurality of plasma cells previously obtained from a test sample from the subject;
comparing the 3D telomeres organization sample signature to a stable SMM 3D telomeres organization reference signature and/or a high-risk SMM 3D telomeres organization reference signature, the stable SMM 3D telomeres organization reference signature defining for one or more of the parameters of a 3D telomeres organization signature associated with stable SMM and the high-risk SMM 3D telomeres organization reference signature defining for one or more of the parameters a 3D telomeres organization signature associated with high-risk SMM;
identifying differences and/or similarities between the 3D telomeres organization test sample signature and the stable SMM 3D telomeres organization reference signature and/or the high-risk SMM 3D telomeres organization reference signature; and
providing the clinical outcome prognosis according to the identified differences and/or similarities, the clinical outcome prognosis being an increased likelihood of stable SMM or an increased likelihood of high-risk SMM, wherein the subject with increased likelihood of high-risk SMM is likely to progress to progress to multiple myeloma (MM).

Embodiment 2. The method of embodiment 1, wherein the test sample is a bone marrow sample, optionally a diagnostic bone marrow biopsy sample or a peripheral liquid biopsy blood sample.

Embodiment 3. The method of embodiment 1 or 2, wherein the prognosis is provided to the subject or the subject's medical professional at time of SMM diagnosis.

Embodiment 4. The method of any one of embodiments 1 to 3, the assaying comprising:
nuclear staining the plurality of the plasma cells by hybridizing the test sample with a labelled probe;
mounting the test sample using an antifade mounting medium;
3D imaging the test sample; and
measuring on the 3D images values for at least one of the telomere parameters to obtain the 3D telomeres organization sample signature.

Embodiment 5. The method of embodiment 4, wherein the labelled probe is a peptide nucleic acid (PNA) probe.

Embodiment 6. The method of embodiment 4 or 5, wherein the 3D imaging comprises acquiring an image dataset of different planes of 3D q-FISH fluorescent signals and reconstructing a 3D image of the telomeres using deconvolution of the images performed with a constrained iterative algorithm, optionally using fluorescence microscopy using a 63×/1.4 oil plan apochromatic objective lens.

Embodiment 7. The method of any one of embodiments 4 to 6, wherein the 3D imaging comprises obtaining a stack of at least 50 images with a sample distance of 200 nm along a z direction and 102 nm in each of a x and a y direction.

Embodiment 8. The method of any one of embodiments 1 to 7, wherein the 3D telomeres organization sample signature is determined on interphase telomeres.

Embodiment 9. The method of any one of embodiments 1 to 8, wherein the 3D telomeres organization sample signature comprises at least two, at least three, at least four, at least five or six of the telomere parameters.

Embodiment 10. The method of any one of embodiments 1 to 9, wherein the comparing comprises comparing the 3D telomeres organization sample signature with a reference 3D telomeres organization signature comprising the telomere numbers, the total telomere intensity, the average telomere intensity, the telomere aggregates, the a/c ratio and/or the nuclear volume, wherein detecting an increase in the telomere numbers, an increase in the total telomere intensity, an increase in the average telomere intensity, an increase in the telomere aggregates, an increase in the a/c ratio and/or an increase in the nuclear volume is indicative of likelihood of SMM with high-risk of progression to MM.

Embodiment 11. The method of any one of embodiments 1 to 10, wherein the stable SMM 3D telomeres organization reference signature is defined by a median or average number of telomeres per cell equal to or less than 38, equal to or less than 34, equal to or less than 32, equal to or less than 30, equal to or less than 28, equal to or less than 26, equal to or less than 24, equal to or less than 22 or equal to or less than 20.

Embodiment 12. The method of any one of embodiments 1 to 10, wherein the high-risk SMM 3D telomeres organization reference signature is defined by a median or average number of telomeres per cell greater than 38, greater than 40, greater than 42, greater than 44, greater than 46, greater than 48 or greater than 50.

Embodiment 13. The method of any one of embodiments 1 to 10, wherein the stable SMM 3D telomeres organization reference signature is defined by a median or average number of aggregates per cell equal to or less than 3.5, equal to or less than 3.0, equal to or less than 2.5, equal to or less than 2.0, equal to or less than 1.5 or equal to or less than 1.0.

Embodiment 14. The method of any one of embodiments 1 to 10, wherein the high-risk SMM 3D telomeres organization reference signature is defined by a median or average number of aggregates per cell greater than 3.5, greater than 4.0, greater than 4.5, greater than 5.0, greater than 5.5 or greater than 6.0.

Embodiment 15. The method of any one of embodiments 1 to 10, wherein the stable SMM 3D telomeres organization reference signature is defined by a median or average a/c ratio per cell equal to or less than 6.5, equal to or less than 6.3, equal to or less than 6.1, equal to or less than 5.9, equal to or less than 5.7, equal to or less than 5.5, equal to or less than 5.3, equal to or less than 5.1 or equal to or less than 4.9.

Embodiment 16. The method of any one of embodiments 1 to 10, wherein the high-risk SMM 3D telomeres organization reference signature is defined by a median or average a/c ratio per cell greater than 6.5 greater than 6.7, greater than 6.9, greater than 7.1, greater than 7.3 or greater than 7.5.

Embodiment 17. The method of any one of embodiments 1 to 10, wherein the stable SMM 3D telomeres organization reference signature is defined by a median or average, average telomere intensity per cell equal to or less than 16,500, equal to or less than 160,000, equal to or less than 15,500, equal to or less than 15,000, equal to or less than 14,500, equal to or less than 14,000, equal to or less than 13,500 or equal to or less than 13,000 fluorescence arbitrary units (f.a.u.).

Embodiment 18. The method of any one of embodiments 1 to 10, wherein the high-risk SMM 3D telomeres organization reference signature is defined by a median or average, average telomere intensity per cell greater than 16,500, greater than 17,000, greater than 17,500, greater than 18,000, greater than 18,500, greater than 19,000, greater than 19,500 or greater than 20,000 f.a.u.

Embodiment 19. The method of any one of embodiments 1 to 10, wherein the stable SMM 3D telomeres organization reference signature is defined by a median or average total telomere intensity per cell equal to or less than 600,000, equal to or less than 550,000, equal to or less than 500,000, equal to or less than 450,000 or equal to or less than 400,000 f.a.u.

Embodiment 20. The method of any one of embodiments 1 to 10, wherein the high-risk SMM 3D telomeres organization reference signature is defined by a median or average total telomere intensity per cell greater than 600,000, greater than 650,000, greater than 700,000, greater than 750,000, greater than 800,000, greater than 850,000, greater than 900,000, greater than 950,000 or greater than 1,00,000 of f.a.u.

Embodiment 21. The method of any one of embodiments 1 to 10, wherein the stable SMM 3D telomeres organization reference signature is defined by a median or average nuclear volume per cell equal to or less than 450,000, equal to or less than 400,000, equal to or less than 350,000, equal to or less than 300,000 or equal to or less than 250,000 μm³.

Embodiment 22. The method of any one of embodiments 1 to 10, wherein the high-risk SMM 3D telomeres organization reference signature is defined by a median or average nuclear volume per cell greater than greater than 450,000, greater than 500,000, greater than 550,000, greater than 600,000, greater than 650,000 or greater than 700,000 μm³.

Embodiment 23. The method of any one of embodiments 11 to 22, wherein the telomere parameter is determined by dividing telomere values into quartiles.

Embodiment 24. The method of any one of embodiments 1 to 23, wherein the subject is a human subject.

Embodiment 25. A method for diagnosing a subject as having stable smoldering multiple myeloma (SMM) or high-risk SMM, comprising:
assaying a plurality of plasma cells using three-dimensional (3D) quantitative fluorescence in situ hybridization (q-FISH) to obtain a 3D telomeres organization sample signature, the 3D telomeres organization sample signature comprising at least one parameter selected from telomere numbers, total telomere intensity, average telomere intensity, telomere aggregates, a/c ratio and nuclear volume, the plurality of plasma cells previously obtained from a test sample from the subject;
comparing the 3D telomeres organization sample signature to a stable SMM 3D telomeres organization reference signature and/or a high-risk SMM 3D telomeres organization reference signature, the stable SMM 3D telomeres organization reference signature defining for one or more of the parameters of a 3D telomeres organization signature associated with stable SMM and the high-risk SMM 3D telomeres organization reference signature defining for one or more of the parameters a 3D telomeres organization signature associated with high-risk SMM;
identifying differences and/or similarities between the 3D telomeres organization test sample signature and the stable SMM 3D telomeres organization reference signature and/or the high-risk SMM 3D telomeres organization reference signature; and
diagnosing according to the identified differences and/or similarities the subject as likely to have stable SMM or as likely to have high-risk SMM.

Embodiment 26. A method for diagnosing a subject as having monoclonal gammopathy of undetermined significance (MGUS), stable smoldering multiple myeloma (SMM) or high-risk SMM, comprising:
assaying a plurality of plasma cells using three-dimensional (3D) quantitative fluorescence in situ hybridization (q-FISH) to obtain a 3D telomeres organization sample signature, the 3D telomeres organization sample signature comprising at least one parameter selected from telomere numbers, total telomere intensity, average telomere intensity, telomere aggregates, a/c ratio and nuclear volume, the plurality of plasma cells previously obtained from a test sample from the subject;
comparing the 3D telomeres organization sample signature to an MGUS 3D telomeres organization reference signature, a stable SMM 3D telomeres organization reference signature and/or a high-risk SMM 3D telomeres organization reference signature, the MGUS 3D telomeres organization reference signature defining for one or more of the parameters of a 3D telomeres organization signature associated with MGUS, the stable SMM 3D telomeres organization reference signature defining for one or more of the parameters of a 3D telomeres organization signature associated with stable SMM and the high-risk SMM 3D telomeres organization reference signature defining for one or more of the parameters of a 3D telomeres organization signature associated with high-risk SMM;
identifying differences and/or similarities between the 3D telomeres organization test sample signature and the MGUS 3D telomeres organization reference signature, the stable SMM 3D telomeres organization reference signature and/or the high-risk SMM 3D telomeres organization reference signature; and
diagnosing according to the identified differences and/or similarities the subject as likely to have MGUS, stable SMM or high-risk SMM.

Embodiment 27. The method of any one of embodiments 1 to 26, wherein when the subject is prognosed or diagnosed as having an increased likelihood of high-risk SMM, the subject is subsequently treated with a therapeutically effective amount of one or more of lenalidomide, dexamethasone, siltuximab, daratumumab, bortezomib, elotuzumab, carfilzomib, thalidomide, cyclophosphamide and combinations thereof.

Embodiment 28. The method of embodiment 27, wherein the subject prognosed or diagnosed as having an increased likelihood of high-risk SMM is subsequently treated with a therapeutically effective amount of bortezomib and dexamethasone.

Embodiment 29. The method of embodiment 27, wherein the subject prognosed or diagnosed as having an increased likelihood of high-risk SMM is subsequently treated with a therapeutically effective amount of siltuximab.

Embodiment 30. The method of embodiment 27, wherein the subject prognosed or diagnosed as having an increased likelihood of high-risk SMM is subsequently treated with a therapeutically effective amount of daratumumab, lenalidomide, bortezomib and dexamethasone.

Embodiment 31. The method of embodiment 27, wherein the subject prognosed or diagnosed as having an increased likelihood of high-risk SMM is subsequently treated with a therapeutically effective amount of elotuzumab.

Embodiment 32. The method of embodiment 27, wherein the subject prognosed or diagnosed as having an increased likelihood of high-risk SMM is subsequently treated with a therapeutically effective amount of carfilzomib, lenalidomide, daratumumab, and dexamethasone, optionally for 3 to 4 cycles as induction therapy or for 8 to 12 cycles for maintenance therapy with a therapeutically effective amount of lenalidomide.

Embodiment 33. The method of embodiment 27, wherein the subject prognosed or diagnosed as having an increased likelihood of high-risk SMM is subsequently treated with a therapeutically effective amount of bortezomib, lenalidomide and dexamethasone.

Embodiment 34. The method of embodiment 27, wherein the subject prognosed or diagnosed as having an increased likelihood of high-risk SMM is subsequently treated with a therapeutically effective amount of bortezomib, thalidomide and dexamethasone.

Embodiment 35. The method of embodiment 27, wherein the subject prognosed or diagnosed as having an increased likelihood of high-risk SMM is subsequently treated with a therapeutically effective amount of bortezomib, cyclophosphamide and dexamethasone.

Embodiment 36. The method of any one of embodiments 1 to 26, wherein when the subject is prognosed or diagnosed as having an increased likelihood of stable SMM, the subject is subsequently monitored.

Embodiment 37. The method of any one of embodiments 1 to 36, wherein the method first comprises obtaining the test sample from the patient.

Embodiment 38. A method of treating a subject with smoldering multiple myeloma (SMM), comprising identifying the subject as likely to have high-risk SMM or stable SMM; and if the subject is identified as likely to have high-risk SMM, administering to the subject a treatment selected from any one of embodiments 27 to 35, and if the subject is identified as likely to have stable SMM, monitoring the subject.

Embodiment 39. A method of providing a personalized treatment plan for a subject with smoldering multiple myeloma, comprising identifying the subject as likely to have high-risk SMM or stable SMM; and providing the subject a treatment plan to be administered to the subject when the subject is identified as having an increased likelihood of high-risk SMM and monitoring the subject identified as having an increased likelihood of stable SMM.

Embodiment 40. The method of embodiment 38 or 39, wherein the subject is identified as likely to have high-risk SMM or stable SMM according to the method of any one of embodiments 1 to 26.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1—Stratification and Risk of Progression in Patients with MGUS, SMM, and MM

### Materials and Methods

### Sample Acquisition and Patient Population

214 patients were recruited between 2010 and 2014. The study population consisted of treatment-naïve patients with MGUS (N=54), SMM (N=24) and MM (N=136). Bone marrow samples were collected at diagnosis. The researchers were blinded to the disease stages of the samples (MGUS, SMM or MM) which were analyzed.

Basic clinical data for the patients are summarized in Table 1.

TABLE 1
Basic clinical characteristics of the study population assessed at time of diagnosis.

| | MGUS | SMM | MM |
|---|---|---|---|
| Number of patients | 54 | 24 | 136 |

Basic Clinical Characteristics

| | | | |
|---|---|---|---|
| Age | 65.7 ± 11.4 | 70.3 ± 13.3 | 67.0 ± 11.5 |
| BMPC (%) | 3.4 ± 1.7 | 16.7 ± 10.2 | 39.0 ± 28.2 |
| M-protein | 6 ± 4.1 | 22.1 ± 19.8 | 26.9 ± 21.4 |
| IgG (%) | 12.6 | 25.5 | 30.8 |
| IgA (%) | 3.6 | 7.7 | 10.1 |
| IgM (%) | 3.3 | 0.7 | 0.3 |
| Lytic lesions (%) | 0/53 (0%) | 0/21 (0%) | 88/134 (65.7%) |

Cytogenetic information

| | | | |
|---|---|---|---|
| Patients with t(11; 14) (%) | 1/36 (2.7%) | N/A | 20/25 (80.0%) |
| Patients with t(4; 14) (%) | 1/8 (12.5) | N/A | 8/19 (42.1%) |
| Patients with del(14q1.3)/13qter (%) | 0/8 (0%) | N/A | 3/19 (15.8%) |

BMPC indicates the degree of bone marrow plasma cell infiltration; M-protein indicates the serum level of myeloma protein. IgG, IgA, and IgM indicate the percentage of patients per cohort with each of the 3 isotypes of immunoglobulin heavy chain as the predominant isotype; n.d. is the percentage of patients whose immunoglobulin isotype data were unavailable. Numbers represent average values along with the maximal variance within them.

The average age of the study population was 65.7 years for MGUS, 70.3 years for SMM, and 67.0 years for MM (Table 1). The average percentage of plasma cell bone marrow infiltration was 3.4%, 16.7%, and 39.0% for MGUS, SMM, and MM, respectively, and the average amount of serum myeloma protein (M-protein) was 6 g/L for MGUS, 22.1 g/L for SMM, and 26.9 g/L for MM. In all groups, the majority of patients had the immunoglobulin G isotype (IgG). A small proportion of all groups had the IgA isotype, followed by IgM subtype. MM was the only group that displayed lytic lesions, as well as a greatly elevated level of serum M-protein compared to MGUS and SMM patients [5]. The most common immunoglobulin isotype in MM patients is IgG, followed by IgA, as previously described [17].

Based on cytogenetic FISH analyses, only two MGUS patients displayed chromosomal aberrations commonly associated with MM. However, cytogenetic data for t(11;14) were only available for 36 patients of the MGUS group and for 25 patients of the MM group. Also, t(4;14) and del(14q1.3)/13qter results were only available for 8 patients of the MGUS group and for 19 of the MM group (Table S1). Interestingly, three MM patients had both t(11;14) and t(4;14) translocations and three other patients had additionally a 14q13 deletion.

### Sample Processing

Bone marrow samples were processed as previously described [17]. The white blood cells were isolated using Ficoll-Paque (GE Healthcare Life Sciences, Baie d'Urfe, Quebec). The cells were washed with Roswell Park Memorial Institute (RPMI) medium (Gibco Life Technologies Inc., Burlington, Ontario) containing 10% fetal bovine serum (FBS) (Gibco Life Technologies Inc.). The cells were placed onto poly-L-lysine coated slides. The cells were then fixed in a 3.7% formaldehyde solution for 20 minutes and the slides were washed twice with 1× phosphate buffered saline (PBS).

### Immunostaining and Telomere Hybridization

The cells were blocked with 4% BSA in 4× saline-sodium citrate (SSC) and incubated with Alexa Fluor® 488 labelled Mouse Anti-Human CD56 antibody (BD Bioscience, San Jose, CA, USA) and Alexa Fluor® 594 labelled anti-human CD138 (Syndecan-1) (Biolegend, San Diego, CA, USA). For telomere hybridization, the cyanine 3 (Cy3)-labeled peptide nucleic acid (PNA) probe (DAKO, Denmark) was used. For probe hybridization, the HYBrite Denaturation and Hybridization System (Vysis; Abbott Diagnostics, Des Plains, IL) was used. Unbound probe was removed by washing in 70% formamide (Sigma-Aldrich, St Louis, MO, USA)/10 mM Tris (pH 7.4) for thirty minutes, in 0.1× saline sodium citrate (SSC) at 55° C. for five minutes, and twice in 2×SSC/0.05% Tween-20 for five minutes. The nuclei were counterstained with 4′,6-diamidino-2-phenylindole (DAPI) (Sigma-Aldrich, St. Louis, Missouri, USA) and mounted with VECTASHIELD (Vector Laboratories, Burlington, Ontario).

### Identification of Malignant Plasma Cells

The malignant plasma cells were differentiated from normal lymphocytes according to their double staining for CD138 conjugated with Alexa Fluor® 594 (BioLegend, San Diego, CA, USA) and CD56 conjugated with Alexa Fluor® 488 (BD Biosciences, San Jose, CA, USA), augmented nuclear size, and weaker DAPI counterstain. Yu et al. (2019) and Klewes et al. (2013) have confirmed that syndecan-1 (CD138) and CD56 positively stained malignant plasma cells, also that malignant plasma cells show larger and weaker DAPI stained nuclei than normal lymphocytes [17,18]. CD138 expression is specific for normal and malignant plasma cells and CD56 (a neural adhesion molecule), a membrane glycoprotein of the immunoglobulin superfamily, is found in 70-80% cases of multiple myeloma (MM) [19].

### Image Acquisition and Nuclear Architecture Analysis

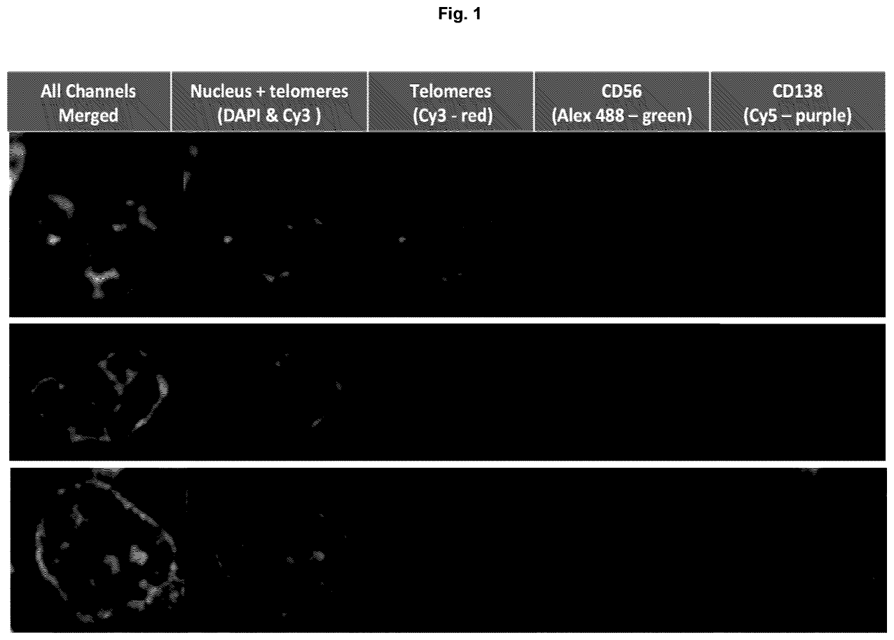

Fifty CD138+/CD56+ interphase nuclei were analyzed per sample. The telomeres were imaged using fluorescence microscopy (Zeiss Axiolmager Z1 microscope (Carl Zeiss, Toronto, Ontario, Canada) equipped with an AxioCam HRm camera, using a 63×/1.4 oil plan apochromatic objective lens). The imaging software ZEN 2.3 software was used for image acquisition, and images were acquired in multichannel mode. 3D imaging of telomeres was performed by acquiring stacks of up to 80 images along the z-axis (specimen depth), with a z-axis step of 0.2 μm between each image in the stack. The sampling distance in both the x- and y-planes was 102 nm. The exposure time for Cy3 (telomeres) was maintained at 100 milliseconds. The images were deconvolved using a constrained iterative algorithm [20]. Representative FISH images are shown in FIG. 1.

### 3D Analysis for Telomeres

After deconvolution, the images were analyzed using TeloView™ v1.03 software (Telo Genomics Corp., Toronto, ON, Canada). TeloView™ determines the following 6 telomere parameters: telomere signal intensity (i.e., length; total and average), number of telomere signals, number of telomere aggregates, nuclear volume, a/c ratio, and distribution of telomeres relative to the nuclear periphery [21]. For each object (e.g., telomere, telomere aggregate) the center of gravity of intensities is determined, and identified by a set of coordinates (x, y, z) denoted by crosses on the computer screen. The integrated intensity of each telomere is calculated.

### Statistical Analyses

For statistical analysis, the software package SAS X version 9.4 (SAS Institute Inc., Cary, NC) was employed to perform nested factorial analysis of variance in the telomere parameters measured using TeloView™. For each case, normally distributed parameters are compared between the two types of cells using nested ANOVA or two-way ANOVA. Multiple comparisons using for example the least square means tests can be used to follow where interaction effects between two factors are found to be significant. Other parameters that are not normally distributed can be compared using a nonparametric Wilcoxon rank sum test. Chi-squared tests were used to compare the percentage of interphase telomere signals at each given intensity level at intervals of 1000 intensity units, ultimately divided into quartiles for analysis. Nested factorial analysis of variance was also used to compare the distribution of signal intensities across MGUS, SMM, and MM. Univariate comparisons for overall survival (OS) were conducted with the log rank test and displayed as Kaplan Meier curves. Cox proportional hazards models were used to estimate hazard ratios (HR) for OS with adjustment for age and diagnosis. A p-value of <0.05 was considered significant.

### Results

Stratification of Patients with MGUS, SMM, and MM.

3D telomere profiles are measured using TeloView™ [26], which assesses the levels of telomere-related genomic instability present in individual nucleus. Specific 3D telomere parameters are associated with disease stability vs. progression [17, 22-26]. Critically short telomeres altered telomere numbers and telomeric aggregates and fusions have all been correlated with increased genomic instability and complexity [17, 25, 27-29].

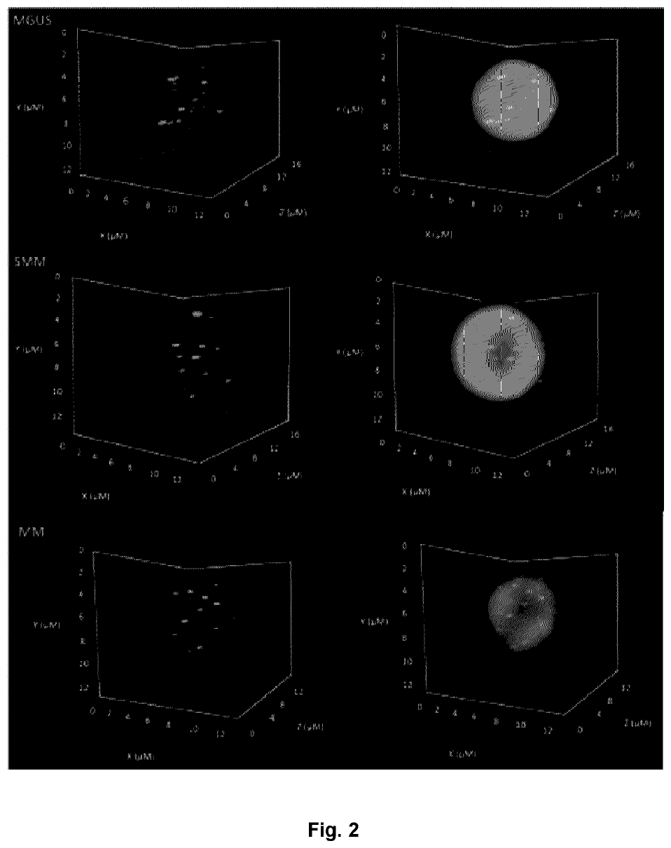

3D telomere profiling was tested to stratify patients with MGUS, SMM or MM. To this end, 3D telomeric profiles of 214 patients (54 MGUS, 24 SMM, and 136 MM) were evaluated using treatment-naïve bone marrow samples at diagnosis. The cells were selected based on their dual positive staining for CD56 and CD138, by their weaker DAPI counterstain, and larger nuclear size when compared to normal lymphocytes In FIG. 2, representative 3D images of CD56+/CD138+ cells from patients in each disease stage are shown. The Cy-3 labelled telomeres appear as light grey signals. Numerous telomere parameters showed alteration along the three disease stages. Most notably, the decrease of telomere number between MM and MGUS, is associated with an increase in genomic instability.

Table 2 summarizes the 3D telomere parameters measured by TeloView™ in MGUS, SMM, and MM as well as in the respective sub-groups of SMM and MM, which include stable SMM and high-risk SMM and stable MM vs. progressive MM, respectively. There were no significant differences in the number of telomere aggregates and number of telomeres per nuclear volume across disease stages (MGUS, SMM, and MM), as seen in Table 2. While MM patients showed less telomere signals than MGUS patients, p=0.0192, no significant differences were found for SMM vs MGUS or MM vs SMM. Next, differences in the total and average intensity of telomere signals were calculated. The average intensity was calculated dividing the total intensity by the total number of telomeres. There was a significant increase (T) in the total intensity of telomere signals in SMM vs MGUS or MM (p=0.0370 and p=0.0019, respectively), although no differences were found between MGUS vs MM (Table 2). The same result was observed in relation to the average telomere intensity (T of average intensity in SMM vs MGUS or MM). Telomere dysfunction can be characterized by different events including telomere shortening and/or telomere aggregate formation [26]. The latter is independent of telomere size or telomerase activity [26]. A decrease of telomere length (represented by average intensity and total intensity) was found, but no significant difference in the number of telomere aggregates was observed, p>0.05. Differences in the a/c ratio and nuclear volume between disease stages were observed. The a/c ratio increased in MM compared to MGUS (p=0.01), pointing to differences in the cell cycle progression between MGUS and MM, but no difference was found for the a/c ratio in MM vs SMM or MGUS vs SMM. The nuclear volume parameter increases in SMM patients compared to MGUS patients, but no significant differences were found between MGUS vs MM or SMM vs MM.

TABLE 2
Significance of telomere parameters in MGUS, SMM and
MM at diagnosis and after five-year follow-up.

| | | | | SMM stable | |
|---|---|---|---|---|---|
| | | | | (stb) vs. | MM stable |
| | | | SMM (all | SMM with | (stb) vs. MM |
| | MGUS vs. | MGUS vs. | cases) vs. | high-risk to | with |
| Telomere | MM (all | SMM (all | MM (all | progression | progressive |
| parameters | cases) | cases) | cases) | (prg) | disease (prg) |
| Telomere | p = 0.0193 | ns | ns | p = <0.0001 | p = <0.0001 |
| numbers | (↑ MGUS × | | | (↓ stb × prg ↑) | (↓ stb × prg ↑) |
| | MM ↓) | | | | |
| Total | Ns | p = 0.0370 | p = 0.0019 | p = <0.0001 | ns |
| telomere | | (↓ MGUS × | (↑ SMM × | (↓ stb × prg ↑) | |
| intensity | | SMM ↑) | MM ↓) | | |
| Average | Ns | p = 0.0009 | p = 0.0097 | p = 0.0493 | p = <0.0001 |
| telomere | | (↓ MGUS × | (↑ SMM × | (↓ stb × prg ↑) | (↑ stb × prg ↓) |
| intensity | | SMM ↑) | MM ↓) | | |
| Telomere | Ns | ns | ns | p = 0.0014 | p = 0.0001 |
| aggregates | | | | (↓ stb × prg ↑) | (↓ stb × prg ↑) |
| a/c ratio | p = 0.0112 | ns | ns | p = <0.0001 | p = <0.0001 |
| | (↓ MGUS × | | | (↓ stb × prg ↑) | (↓ stb × prg ↑) |
| | MM ↑) | | | | |
| Nuclear | Ns | p = 0.05 | ns | p = 0.0033 | p = <0.0001 |
| volume | | (↓ MGUS × | | (↓ stb × prg ↑) | (↓ stb × prg ↑) |
| | | SMM ↑) | | | |
| Telomeres | Ns | ns | ns | ns | ns |
| per nuclear | | | | | |
| volumes | | | | | |

ns: not significantSubgrouping within SMM and MM patients highlights risk and progression groups

Myeloma and its precursor lesions are heterogeneous diseases. Therefore, it was important to stratify the patients of the SMM and MM based on their 5-year clinical information (progression and survival) as recorded in the clinical follow-up. Patients without follow-up were excluded, limiting the analysis to a total of 20 SMM patients. Fifteen of the 20 SMM patients remained stable for over 5 years, while five progressed to full stage multiple myeloma within 1 to 3 years from point of diagnosis. They were stratified as SMM-stable (or indolent SMM) and SMM-progression (or high-risk SMM), respectively. The disease progression of high-risk SMM patients was confirmed clinically by MM caused morbidity. Comparison of the 3D telomere profiles between SMM-stable and SMM-progression showed the presence of two groups with different levels of telomere related genomic instability. As shown in Table 3 below, telomere parameter values in stable SMM patients and high-risk SMM patients are obtained by dividing telomere values into quartiles for analysis. Five telomere parameters were highlighted different, namely telomere numbers, intensity (total and average), telomere aggregates, a/c ratio and nuclear volume (Table 2).

TABLE 3
3D telomere parameters for Stable SMM versus High-risk SMM

| | Variable | | | | | |
|---|---|---|---|---|---|---|
| | Stable SMM Patients | | | High-risk SMM Patients | | |
| Quartile | 25% | 50% | 75% | 25% | 50% | 75% |
| Telomere numbers | 31 | 37 | 42 | 33 | 41 | 48 |
| (per cell) | | | | | | |
| Average telomere intensity | 13,259 | 16,084 | 19,068 | 14,375 | 16,714 | 19,843 |
| (f.a.u .* per cell) | | | | | | |
| Total telomere intensity | 453,178 | 580,171 | 713,662 | 548,170 | 666,166 | 801,713 |
| (f.a.u. per cell) | | | | | | |
| Telomere aggregates | 2 | 3 | 5 | 3 | 4 | 6 |
| (per cell) | | | | | | |
| a/c ratio | 4.02 | 5.06 | 6.86 | 6.46 | 8.69 | 12.02 |
| (per cell) | | | | | | |
| Nuclear volume | 297,419 | 387,029 | 499,290 | 283,863 | 483,561 | 689,191 |
| (μm³ per cell) | | | | | | |

*f.a.u. = fluorescence arbitrary units

For the MM group, where no patients were excluded due to the absence of clinical follow up, 47 deaths related to the disease were observed leading to 89 MM patients with stable disease and 47 MM patients with progressive disease. As for the telomere per nuclear volume parameter, it continued to show no significance. Total intensity was also not significant between subgroups (stable vs progression). However, when other telomere parameters were compared, namely total number of signals, average intensity, telomere aggregates, nuclear volume and a/c ratio, all p values are highly significant. MM patients with progressive disease have increased (T) telomere signals, telomere aggregates, a/c ratio and nuclear volume with a decrease in average intensity of telomere signals in comparison with MM patients with stable disease (Table 2).

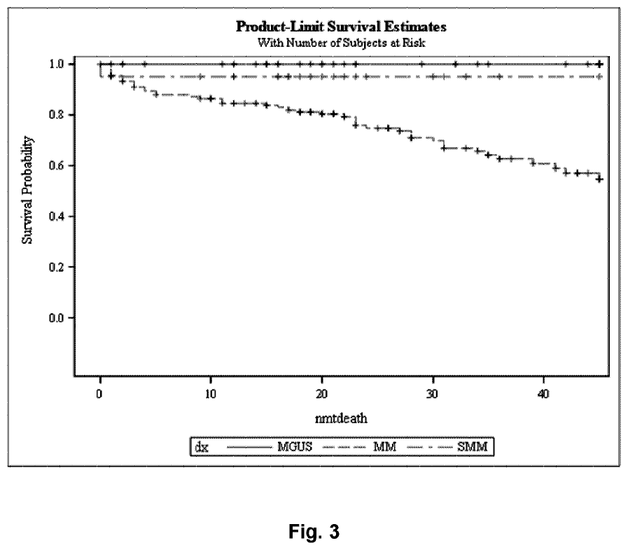

It was next investigated whether any of the telomere parameters was of prognostic value on overall survival (OS) by performing cox's proportional hazards modeling in the cohort. 8 patients without clinical follow-up were excluded from this analysis. Kaplan-Meier survival analysis (performed using the log-rank test) showed agreement with previous reports, where MM patient survival was significantly inferior to SMM and MGUS patients (FIG. 3). Adjustment for multiple comparisons is shown in Table 4, wherein “Raw” refers to p-values without adjustments for multiple comparisons, and “Sidak” refers to p-values according to the Sidak adjustment for multiple comparisons.

TABLE 4
Adjustment for multiple comparisons for the log-
rank test in patients with MGUS, SMM, and MM.

p-Values

| Comparison | | χ² | Raw | Sidak |
|---|---|---|---|---|
| MGUS | MM | 23.6401 | <0.0001 | <0.0001 |
| MGUS | SMM | 8.6934 | 0.0032 | 0.0096 |
| MM | SMM | 18.6091 | <0.0001 | <0.0001 |

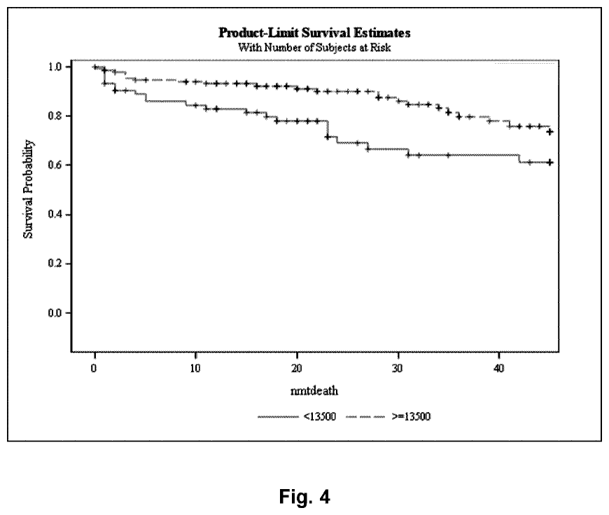

When considering a telomere average intensity threshold (<13500 fluorescent arbitrary units (f.a.u.)), a subset of MM patients with inferior survival was identified (FIG. 4). Average intensity is highly prognostic in multiple myeloma (MM). Patients with a telomere average intensity below 13500 a.u. had significantly shorter OS when compared to patients above 13500 a.u. Adjustment for multiple comparisons is shown in Table 5, wherein “Raw” refers to p-values without adjustments for multiple comparisons, and “Sidak” refers to p-values according to the Sidak adjustment for multiple comparisons. The proportional hazard modeling showed that total intensity and average intensity were significant parameters to predict OS in MM. In multivariate analysis, every predictor was adjusted for the other predictors in the model (Table 6).

TABLE 5
Adjustment for multiple comparisons for the log-rank test
when considering telomere average intensity threshold.

Comparison

| | | |
|---|---|---|
| Average | Average | p-Values |

| intensity | Intensity | χ² | Raw | Sidak |
|---|---|---|---|---|
| <13500 | ≥13500 | 6.1106 | 0.0134 | 0.0134 |

TABLE 6
Cox's proportional hazards modeling of telomere signals adjusted by age and diagnosis.
Analysis of Maximum Likelihood Estimates

| | | | | | | | 95% Hazard | |
|---|---|---|---|---|---|---|---|---|
| | | | | | | | Ratio | |
| | | Parameter | Standard | Chi- | | Hazard | Confidence | |
| Parameter | DF | Estimate | Error | Square | Pr > ChiSq | Ratio | Limits | |
| Avint | 1 | −0.45436 | 0.13470 | 11.3781 | 0.0007 | 0.635 | 0.488 | 0.827 |
| Totin | 1 | 0.04296 | 0.02112 | 4.1358 | 0.0420 | 1.044 | 1.002 | 1.088 |
| Mmdx | 1 | 2.07806 | 0.60041 | 11.9789 | 0.0005 | 7.989 | 2.463 | 25.916 |
| Smmdx | 1 | 2.01007 | 0.75049 | 7.1736 | 0.0074 | 7.464 | 1.715 | 32.492 |
| Age | 1 | 0.04876 | 0.01310 | 13.8605 | 0.0002 | 1.050 | 1.023 | 1.077 |

The measure of effect is the hazard ratio, which is the risk of failure (i.e., the risk or probability of suffering the event in question). If the hazard ratio is less than 1, then the predictor is protective (i.e., associated with improved survival). On the other hand, if the hazard ratio is greater than 1, then the predictor is associated with increased risk (or decreased survival). The p value shows statistically significant associations between the first column parameters with mortality. DF—degree of freedom, each predictor occupies 1 degree of freedom in the model. Avint—average intensity of telomere signals; totin—total intensity of telomere signals; mmdx—MM diagnosis; and smmdx—SMM diagnosis. It is important that to note that effects are adjusted for all predictors in the model. For total intensity, the HR was 1.0005 [95% confidence interval (CI), 1.000-1.009], p=0.0297, and for average intensity, the HR was 0.594 [95% confidence interval (CI), 0.467-0.754], p<0.0001. Then, after adjustment for age and diagnosis, both total intensity (totin) and average intensity (avint), summarized in Table 2, continued to be associated with significantly shorter survival. Adjustment for age alone had total intensity HR 1.005, 95% CI 1.000-1.009, p=0.04, and average intensity HR 0.602, 95% CI 0.463-0.784, p=0.0002; while adjustments for age and diagnosis had total intensity HR 1.044, 95% CI 1.000-1.008, p=0.04, and average intensity HR 0.635, 95% CI 0.488-0.827, p=0.0007).

Average Telomere Intensity and Number of Telomere Aggregates are Significantly Associated with Shorter Overall Survival of MM Patients

Furthermore, based on the notion that SMM is a not a unique biological entity but a stage in the progression of tumor plasma cells, leading to symptomatic MM, two new groups (MGUS and MM) were created. The stable SMM patients were merged with the MGUS group (MGUS-like) and the high-risk SMM patients were combined with the MM group (MM-like). In order to assess if the new grouping displayed any prognostic impact in overall survival, multivariate analysis was performed. The proportional hazard modeling showed that total intensity and average intensity continued to be significant parameters to predict overall survival in MM. After adjustment for age and diagnosis, total intensity and average intensity continued to be associated with significantly shorter survival, total intensity HR 1.044, 95% CI 1.002-1.0888, p=0.04, and average intensity HR 0.635, 95% CI 0.488-0.827, p=0.0007.

### Discussion

3D telomere analysis measures the level of genomic instability, and specific telomere profiles are associated with disease stability vs. progression [17, 21-25]. Extremely short telomeres, telomere dysfunction, and fusions have all been correlated with disease progression. Therefore, telomere profiling may represent both a clinically useful prognostic tool and a potential guide for therapeutic intervention [23]. In current clinical practice, patients diagnosed with MGUS or SMM are monitored but not treated. As described above, bone marrow samples from a cohort of 214 patients diagnosed with MGUS (54), SMM (24), and MM (136), with clinical follow-up of a minimum of 50 months, were evaluated. Several significant differences in the telomere parameters of the three disease stages were found. In the SMM patient group, five different telomere parameters identified patients with stable or progressive disease and allowed to stratify this group of SMM patients into high-risk SMM versus low-risk SMM. Similar results were observed for MM patients. This risk stratification has the potential to guide evidence-based treatment decisions of SMM patients with a high risk of progression or MM patients with active disease.

In Cox's proportional hazards analysis the average intensity and total intensity associated with shorter overall survival in MM (FIG. 4 and Table 6). Setting as threshold of 13.500 a.u for average intensity provided a clear differential OS of patients below and above the set threshold in the survival curve (FIG. 4). A decrease of telomere length in MM compared to normal controls has been described in other studies. Wu et al. (2003) investigated telomere length in CD138+ flow sorted cells from bone marrow of 115 MM patients (newly diagnosed or relapsed) and 7 healthy donors [30]. The results showed significantly reduced telomere length in MM patients compared to the telomere length in plasma cells from healthy donors [30]. Cottliar et al. (2003) studied bone marrow (BM) cells from 31 patients with MM and 2 with MGUS. They also observed reduction in telomere length in MM patient samples at diagnosis and during relapse, but they noticed that telomere length (in BM cells) was restored after disease remission [31]. Nevertheless, as discussed previously, the three aforementioned studies' limitation was the use of a pool of CD138+ cells. In contrast, the results were achieved through single cell analysis, which requires less patient sample material while providing a better assessment of clonal diversity and variability in the MM cell population.

Interestingly, different telomere parameters differentiated MGUS from SMM or MM, but no common parameter emerged for the whole disease spectrum. This lack of a common parameter could be due to disease heterogeneity and clonal evolution at each stage. When Bolli et al. (2018) characterized the genomic landscape of high-risk SMM using whole genome sequencing, they observed that cytogenetic, mutational, and rearrangement profiles were very similar to those described for MM [35]. The data shows two distinct subpopulations inside of SMM and MM groups associated with a more aggressive disease, which is an important finding, since SMM patients currently are not usually treated until clinical symptoms of progression to MM appear. The subpopulation groups of each of these two stages of the disease were associated with different levels of genomic instability. This stratification has the potential to identify SMM and MM patients that will benefit from immediate treatment decisions.

### Example 2—Model for Risk of Disease Progression of Individual Smoldering Myeloma Patients with Over 80% Accuracy

A cohort of 88 SMM patients was analyzed using TeloView technology. Sample processing, imaging, and image processing were done in accordance with the materials and methods described in Example 1, as appropriate. The cohort for this study included 29 SMM patients who progressed to full stage MM within 2 years (high-risk SMM) and 59 SMM patients who remained in the SMM stage for over 5 years (stable SMM). Biological samples were collected from all patients at point of diagnosis, and the patient follow up data was known. The study was conducted retrospectively, but patient data was deidentified, and analysis was conducted blinded to patient outcome. Initial univariate analysis using simple nested methodologies that accounted for individual patient inter-variability identified four telomeric parameters measured by TeloView as potential predictors suitable for regression modeling; a/c ratio, nuclear volume, nuclear telomere distribution, and total telomere length. A summary of the nested ANOVA and t-test statistical analysis is shown in Table 7.

TABLE 7
The significant progression predictors and their respective p-values

| | | Equality of |
|---|---|---|
| Telomere Parameter | p-value | variance |
| Nuclear telomere distribution | 0.02 | <0.0001 |
| a/c ratio | 0.001 | <0.0001 |
| Nuclear volume | 0.03 | <0.001 |
| Total telomere length | 0.03 | <0.0001 |

### Multivariate Modeling

Multivariate analysis was conducted using identified telomere predictors with forward and backward stepwise selection methodologies, in receiver operating characteristic (ROC) modeling, using the data of the 88 patients as a training dataset. From the bivariate analysis of the two groups (i.e., stable SMM and high-risk SMM), a list of predictors was compiled. These predictors were found to discriminate between the two groups generally, using a significance level of p<0.30. As these predictors may come from the same parameter, though aggregated differently (e.g., 25th percentile, 50th percentile, 75th percentile, mean, and inter-quartile ranges—distance from 25th percentile to 75th percentile) from the cells studied, such highly correlated predictors were avoided by including them separately in the stepwise analysis.

Two types of stepping procedures were used to analyze the potential predictors—forward stepwise and backward stepwise, additionally predictive predictors were added forward or deleted backward with level set at p=0.06 to select and p=0.05 to stay. The models found to meet fit Akaike information criterion (AIC) as well as global likelihood ratio tests were selected, and their receiver operating characteristic (ROC) curves (sensitivity and specificity points) plotted. An estimate was made for each predictor in the beta coefficients, odds ratios along with their confidence intervals. These coefficients were adjusted for the other predictors in the model.

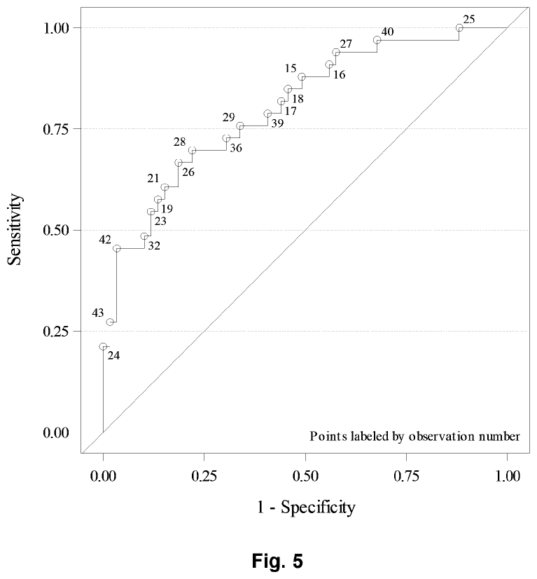

The output of the ROC was used to select the sensitivity and specificity that best serve clinical utility. Several predictive classification models were achieved (Table 8), wherein the best one (Table 8, top row) has an area-under-the-curve (AUC) of 0.80 (thus an accuracy of 80%) with a possible sensitivity and specificity of 75% and 70%, respectively (FIG. 5). The best ROC scoring model included three of the telomere predictors that were identified in the univariate stage of the analysis, namely nuclear telomere distribution, the a/c ratio, and the total telomere length.

TABLE 8
Area-under-the-curve (AUC) of predictive models

| Predictors | Corresponding AUC |
|---|---|
| 3 predictors: nuclear telomere distribution, | 0.80 |
| a/c ratio & total telomere length | |
| 2 predictors: A/C ratio & telomere numbers | 0.75 |
| 1 predictor: A/C ratio only | 0.64 |
| 2 predictors: A/C ratio & total number of telomere | 0.77 |
| aggregates | |
| 2 predictors: A/C ratio & nuclear telomere | 0.78 |
| distribution | |

The results of this study present an independent prognostic biomarker able to stratify SMM patients into their respective risk groups, allowing for evidence-based treatment decision of high risk SMM patients while minimizing unnecessary treatment in stable SMM patients. Equally important, these results present a means to confidently monitor low risk SMM patients to confirm the stability of the SMM disease over time.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### REFERENCES

1. Hallek M, Bergsagel P L, Anderson K C. Multiple myeloma: increasing evidence for a multistep transformation process. Blood. 1998 Jan. 1; 91(1):3-21.
2. Dhodapkar M V1. MGUS to myeloma: a mysterious gammopathy of underexplored significance. Blood. 2016 Dec. 8; 128(23):2599-2606.
3. Albagoush S A, Azevedo A M. Cancer, Multiple Myeloma. StatPearls [Internet]. Treasure Island (FL): StatPearls Publishing; 2019 January-2019 Mar. 19. [BOOK}
4. Semin Oncol. 2019 April; 46(2):112-120. doi: 10.1053/j.seminoncol.2019.03.001. Epub 2019 Apr. 24. State of the science in smoldering myeloma: Should we be treating in the clinic? Hill E1, Dew A2, Kazandjian D3.
5. Kyle R A, Remstein E D, Therneau T M, et al: Clinical course and prognosis of smoldering (asymptomatic) multiple myeloma. N Engl J Med 2007; 356: pp. 2582-2590.
6. Ziogas D C1, Dimopoulos M A1, Kastritis E1. Expert Rev Hematol. 2018 November; 11(11):863-879. doi: 10.1080/17474086.2018.1537776. Epub 2018 Oct. 26. Prognostic factors for multiple myeloma in the era of novel therapies.
7. Greipp, P. R., San Miguel, J., Durie, B. G., Crowley, J. J., Barlogie, B., Blade, J., Boccadoro, M., Child, J. A., Avet-Loiseau, H., Kyle, R. A., Lahuerta, J. J., Ludwig, H., Morgan, G., Powles, R., Shimizu, K., Shustik, C., Sonneveld, P., Tosi, P., Turesson, I. & Westin, J. (2005) International staging system for multiple myeloma. Journal of Clinical Oncology, 23, 3412-3420.
8. Cagnetta A1, Lovera D1, Grasso R1, Colombo N1, Canepa L1, Ballerini F1, Calvio M1, Miglino M1, Gobbi M1, Lemoli R1, Cea M1. Mechanisms and Clinical Applications of Genome Instability in Multiple Myeloma. Biomed Res Int. 2015; 2015:943096. doi: 10.1155/2015/943096. Epub 2015 Oct. 22.
9. Bolli N., Avet-Loiseau H., Wedge D. C., et al. Heterogeneity of genomic evolution and mutational profiles in multiple myeloma. Nature Communications. 2014; 5, article 2997 doi: 10.1038/ncomms3997.
10. Neri P., Bahlis N. J. Genomic instability in multiple myeloma: mechanisms and therapeutic implications. Expert Opinion on Biological Therapy. 2013; 13(supplement 1):S69-S82. doi: 10.1517/14712598.2013.814637.
11. Morgan G. J., Walker B. A., Davies F. E. The genetic architecture of multiple myeloma. Nature Reviews Cancer. 2012; 12(5):335-348. doi: 10.1038/nrc3257.
12. Gadji M1, 2, Pozzo A R1. From cellular morphology to molecular and epigenetic anomalies of myelodysplastic syndromes. Genes Chromosomes Cancer. 2019 July; 58(7):474-483. doi: 10.1002/gcc.22689. Epub 2018 Nov. 18
17. Yu P L I, Wang Y, Tammur P, Tamm A, Punab M, Rangel-Pozzo A, Mai S. Distinct Nuclear Organization of Telomeres and Centromeres in Monoclonal Gammopathy of Undetermined Significance and Multiple Myeloma. Cells. 2019 Jul. 15; 8(7). pii: E723. doi: 10.3390/cells8070723.
18. Klewes L, Vallente R, Dupas E, Brand C, Grün D, Guffei A, Sathitruangsak C, Awe J A, Kuzyk A, Lichtensztejn D, Tammur P, Ilus T, Tamm A, Punab M, Rubinger M, Olujohungbe A, Mai S. Three-dimensional Nuclear Telomere Organization in Multiple Myeloma. Transl Oncol. 2013 Dec. 1; 6(6):749-56. eCollection 2013 Dec. 1.
19. Miyazaki K, Suzuki K. CD56 for Multiple Myeloma: Lack of CD56 May Be Associated with Worse Prognosis. Acta Haematol. 2018; 140(1):40-41. doi: 10.1159/000490231. Epub 2018 Aug. 15.
20. Schaefer, L. H.; Schuster, D.; Herz, H. Generalized approach for accelerated maximum likelihood based image restoration applied to three-dimensional fluorescence microscopy. Journal of microscopy 2001, 204, 99-107, DOI: 10.1046/j.1365-2818.2001.00949.x
21. Jones C H1, Pepper C, Baird D M. Telomere dysfunction and its role in haematological cancer. Br J Haematol. 2012 March; 156(5):573-87. doi: 10.1111/j.1365-2141.2011.09022.x. Epub 2012 Jan. 11.
22. Wark L, Quon H, Ong A, Drachenberg D, Rangel-Pozzo A, Mai S. Long-Term Dynamics of Three Dimensional Telomere Profiles in Circulating Tumor Cells in High-Risk Prostate Cancer Patients Undergoing Androgen-Deprivation and Radiation Therapy. Cancers (Basel). 2019 Aug. 14; 11(8). pii: E1165. doi: 10.3390/cancers11081165.
23. Drachenberg D, Awe J A, Rangel Pozzo A, Saranchuk J, Mai S. Advancing Risk Assessment of Intermediate Risk Prostate Cancer Patients. Cancers (Basel). 2019 Jun. 20; 11(6). pii: E855. doi: 10.3390/cancers11060855.
24. Rangel-Pozzo A, Corrêa de Souza D, Schmid-Braz A T, de Azambuja A P, Ferraz-Aguiar T, Borgonovo T, Mai S. 3D Telomere Structure Analysis to DetectGenomic Instability and Cytogenetic Evolution in Myelodysplastic Syndromes. Cells. 2019 Apr. 2; 8(4). pii: E304. doi: 10.3390/cells8040304.
25. Mai S1, Garini Y. The significance of telomeric aggregates in the interphase nuclei of tumor cells. J Cell Biochem. 2006 Apr. 1; 97(5):904-15.
26. Vermolen, B. J.; Garini, Y.; Mai, S.; Mougey, V.; Fest, T.; Chuang, T. C.-Y.; . . . Young, I. T. Characterizing the three-dimensional organization of telomeres. Cytometry Part A 2005, 67: 144-150, DOI: 10.1002/cyto.a.20159
27. Wu K D1, Orme L M, Shaughnessy J Jr, Jacobson J, Barlogie B, Moore M A. Telomerase and telomere length in multiple myeloma: correlations with disease heterogeneity, cytogenetic status, and overall survival. Blood. 2003 Jun. 15; 101(12):4982-9. Epub 2003 Feb. 27.
28. Cottliar A1, Pedrazzini E, Corrado C, Engelberger M I, Narbaitz M, Slavutsky I. Telomere shortening in patients with plasma cell disorders. Eur J Haematol. 2003 November; 71(5):334-40.
29. Walker B A #1, Wardell C P #1, Melchor L1, Brioli A1, 2, Johnson D C1, Kaiser M F1, Mirabella F1, Lopez-Corral L3, Humphray S4, Murray L4, Ross M4, Bentley D4, Gutiêrrez N C3, Garcia-Sanz R3, San Miguel J3, Davies F E5, Gonzalez D1, Morgan G J1. Intraclonal heterogeneity is a critical early event in the development of myeloma and precedes the development of clinical symptoms. Leukemia. 2014 February; 28(2):384-390. doi: 10.1038/leu.2013.199. Epub 2013 Jul. 2.
30. Cagnetta A1, Lovera D1, Grasso R1, Colombo N1, Canepa L1, Ballerini F1, Calvio M1, Miglino M1, Gobbi M1, Lemoli R1, Cea M1. Mechanisms and Clinical Applications of Genome Instability in Multiple Myeloma. Biomed Res Int. 2015; 2015:943096. doi: 10.1155/2015/943096. Epub 2015 Oct. 22.
31. Capp J P1, Bataille R2. Multiple Myeloma Exemplifies a Model of Cancer Based on Tissue Disruption as the Initiator Event. Front Oncol. 2018 Sep. 10; 8:355. doi: 10.3389/fonc.2018.00355. eCollection 2018.
32. Bolli N, Maura F, Minvielle S, Gloznik D, Szalat R, Fullam A, Martincorena I, Dawson K J, Samur M K, Zamora J, Tarpey P, Davies H, Fulciniti M, Shammas M A, Tai Y T, Magrangeas F, Moreau P, Corradini P, Anderson K, Alexandrov L, Wedge D C, Avet-Loiseau H, Campbell P, Munshi N. 1. Genomic patterns of progression in smoldering multiple myeloma. Nat Commun. 2018 Aug. 22; 9(1):3363. doi: 10.1038/s41467-018-05058-y.
33. González, D.; van der Burg, M.; Garcia-Sanz, R.; Fenton, J. A.; Langerak, A. W.; González, M.; . . . Morgan, G. J. Immunoglobulin gene rearrangements and the pathogenesis of multiple myeloma. Blood 2007, 110, 3112-3121, DOI: 10.1182/blood-2007-02-069625
34. Kaelin W. G., Jr. The concept of synthetic lethality in the context of anticancer therapy. Nature Reviews Cancer. 2005; 5(9):689-698. doi: 10.1038/nrc1691. [PubMed] [CrossRef][Google Scholar]
35. Neri P., Ren L., Gratton K., et al. Bortezomib-induced ‘BRCAness’ sensitizes multiple myeloma cells to PARP inhibitors. Blood. 2011; 118(24):6368-6379. doi: 10.1182/blood-2011-06-363911.
36. Botrugno O A1, Bianchessi S1, Zambroni D1, Frenquelli M1, Belloni D1, Bongiovanni L1, Girlanda S1, Di Terlizzi S1, Ferrarini M1, Ferrero E1, Ponzoni M1, Marcatti M1, Tonon G2. ATR addiction in multiple myeloma: synthetic lethal approaches exploiting established therapies. Haematologica. 2019 Nov. 14. pii: haematol.2018.215210. doi: 10.3324/haematol.2018.215210.
37. Bajpai R1, Matulis S M1, Wei C1, Nooka A K1, Von Hollen H E1, Lonial S1, Boise L H1, Shanmugam M1. Targeting glutamine metabolism in multiple myeloma enhances BIM binding to BCL-2 eliciting synthetic lethality to venetoclax. Oncogene. 2016 Jul. 28; 35(30):3955-64. doi: 10.1038/onc.2015.464. Epub 2015 Dec. 7.
38. Padala S A, Barsouk A, Barsouk A, Rawla P, Vakiti A, Kolhe R, Kota V, Ajebo G H. Epidemiology, Staging, and Management of Multiple Myeloma. Med Sci (Basel). 2021 Jan. 20; 9(1):3. doi: 10.3390/medsci9010003. PMID: 33498356; PMCID: PMC7838784.
39. Rajkumar S V, Kumar S, Lonial S, Mateos M V. Smoldering multiple myeloma current treatment algorithms. Blood Cancer J. 2022 Sep. 5; 12(9):129. doi: 10.1038/s41408-022-00719-0.
40. Lussier T, Schoebe N, Mai S. Risk Stratification and Treatment in Smoldering Multiple Myeloma. Cells. 2021 Dec. 31; 11(1):130. doi: 10.3390/cells11010130.
41. Mateos M V, Hernández M T, Salvador C, de la Rubia J, de Arriba F, Luciâ Lôpez-Corral L, Rosiñol L, Paiva B, Palomera L, Bargay J, Oriol A, Prosper F, Lôpez J, Arguiñano J M, Bladé J, Lahuerta J J, San-Miguel J. Lenalidomide-dexamethasone versus observation in high-risk smoldering myeloma after 12 years of median follow-up time: A randomized, open-label study. Eur J Cancer. 2022 October; 174:243-250. DOI: 10.1016/j.ejca.2022.07.030.
42. Mathur S, Glogowska A, McAvoy E, Righolt C, Rutherford J, Willing C, Banik U, Ruthirakuhan M, Mai S, Garcia A. Three-dimensional quantitative imaging of telomeres in buccal cells identifies mild, moderate, and severe Alzheimer's disease patients. J Alzheimers Dis. 2014. 39(1):35-48. doi: 10.3233/JAD-130866

1. A method of clinical outcome prognosis or of diagnosis, comprising:
assaying a plurality of plasma cells using three-dimensional (3D) quantitative fluorescence in situ hybridization (q-FISH) and obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model consisting of the telomere parameters: a) nuclear telomere distribution, a/c ratio, and total telomere length; b) a/c ratio and telomere numbers; c) a/c ratio and nuclear telomere distribution, or d) a/c ratio and telomere aggregates; and
providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years.

2. The method of claim 1, wherein the test sample is a bone marrow sample or a blood sample, optionally a diagnostic bone marrow biopsy sample or a peripheral liquid biopsy blood sample.

3. The method of claim 1, wherein the prognosis or the diagnosis is provided to the subject or the subject's medical professional at time of SMM diagnosis.

4. The method of claim 1, the assaying comprising:
labelling nuclei of the plurality of the plasma cells with a fluorescent nuclear stain or probe, optionally wherein the fluorescent nuclear stain is 4′,6-diamidino-2-phenylindole (DAPI);
tagging telomeres in the plurality of plasma cells through in situ hybridization with a telomere-specific labelled probe, optionally a peptide nucleic acid (PNA) probe,
mounting the test sample using an antifade mounting medium;
3D imaging the test sample; and
measuring on the 3D images values for the at least one telomere parameter to obtain the 3D telomere organization sample signature.

5. The method of claim 4, wherein assaying further comprises tagging peptide CD138 in the plurality of plasma cells with a CD138-specific antibody linked to a fluorescent label and/or tagging peptide CD56 in the plurality of plasma cells with a CD56-specific antibody linked to a fluorescent label prior to the mounting of the test sample.

6. The method of claim 4, wherein the 3D imaging comprises acquiring an image dataset of different planes of 3D q-FISH fluorescent signals and reconstructing a 3D image of the telomeres using deconvolution of the images performed with a constrained iterative algorithm, optionally using fluorescence microscopy and/or obtaining a stack of at least 50 images with a sample distance of 200 nm along a z direction and 102 nm in each of a x and a y direction.

7. The method of claim 1, wherein the 3D telomere organization sample signature is determined from interphase plasma cells.

8. The method of claim 1, wherein the one or more of and/or the at least telomere parameter comprises an absolute value, a mean, a median, a ratio, a percentile, a quartile, a rank, a range, or a combination thereof.

9. The method of claim 1, wherein the sample is a diagnostic sample.

10. The method of claim 1, wherein the one or more of the at least one telomere parameter of the classification model is selected to have an accuracy of at least at least 75%, at least 80%, at least 85%, at least 90%, at least 95%, at least 96%, at least 97%, least 98%, at least 99% or 100% in distinguishing between stable SMM and high-risk SMM.

11. The method of claim 1, wherein the subject is a human subject.

12. The method of claim 1, wherein when the subject is prognosed or diagnosed as having an increased likelihood of high-risk SMM, the subject is subsequently treated with one or more of lenalidomide, dexamethasone, siltuximab, daratumumab, bortezomib, elotuzumab, carfilzomib, thalidomide, cyclophosphamide and combinations thereof.

13. The method of claim 1, wherein the subject prognosed or diagnosed as having an increased likelihood of high-risk SMM is subsequently treated with: a) bortezomib and dexamethasone; b) siltuximab, c) daratumumab, lenalidomide, bortezomib and dexamethasone, d) elotuzumab, carfilzomib, lenalidomide, daratumumab, and dexamethasone, optionally for 3 to 4 cycles as induction therapy, e) bortezomib, lenalidomide and dexamethasone, f) bortezomib, thalidomide and dexamethasone, g) bortezomib, cyclophosphamide and dexamethasone, h) lenalidomide, or i) lenalidomide and dexamethasone.

14. The method of claim 1, wherein when the subject is prognosed or diagnosed as having an increased likelihood of stable SMM or as having an increased likelihood of high-risk SMM, the subject is subsequently monitored.

15. A method of monitoring a subject prognosed or diagnosed as having an increased likelihood of stable SMM or as having an increased likelihood of high-risk SMM, comprising:
obtaining a subsequent sample from the subject, the subsequent sample comprising a plurality of plasma cells;
assaying the plurality of plasma cells according to the assaying step of claim 1, to obtain a 3D telomere organization monitoring signature;
comparing the 3D telomere organization monitoring signature to a 3D telomere organization signature obtained from a previous sample from the subject; and
providing an updated clinical outcome prognosis or an updated diagnosis to the subject having an increased likelihood of stable SMM, wherein the updated clinical outcome prognosis or the updated diagnosis is of no change, an increased likelihood of stable SMM compared to the previous sample, or an increased likelihood of high-risk SMM, or providing an updated clinical outcome prognosis, an updated diagnosis, or an evaluation of treatment response to the subject having an increased likelihood of high-risk SMM, wherein the updated prognosis or diagnosis or the evaluation is an amelioration, stabilization, or worsening of SMM or a symptom thereof.

16. The method of any one of claim 1, wherein the method first comprises obtaining the test sample from the patient.

17. A method of treating a subject with smoldering multiple myeloma (SMM) based on a 3D telomere organization signature from the subject, comprising administering to the subject a treatment selected from claim 12 when the subject has an increased likelihood of high-risk SMM, or monitoring the subject when the subject has an increased likelihood of stable SMM.

18. A method of providing a personalized treatment plan for a subject with smoldering multiple myeloma (SMM) based on a 3D telomere organization sample signature of the subject, comprising providing the subject the personalized treatment plan to be administered to the subject when the subject has an increased likelihood of high-risk SMM or monitoring the subject when the subject has an increased likelihood of stable SMM determined according to the method of claim 1, wherein the treatment plan comprises a treatment selected from one or more of lenalidomide, dexamethasone, siltuximab, daratumumab, bortezomib, elotuzumab, carfilzomib, thalidomide, cyclophosphamide and combinations thereof, optionally a) bortezomib and dexamethasone; b) siltuximab, c) daratumumab, lenalidomide, bortezomib and dexamethasone, d) elotuzumab, carfilzomib, lenalidomide, daratumumab, and dexamethasone, optionally for 3 to 4 cycles as induction therapy, e) bortezomib, lenalidomide and dexamethasone, f) bortezomib, thalidomide and dexamethasone, q) bortezomib, cyclophosphamide and dexamethasone, h) lenalidomide, or i) lenalidomide and dexamethasone, when the subject is prognosed or diagnosed as having an increased likelihood of high-risk SMM.

19. The method of claim 17, wherein the subject having an increased likelihood of high-risk SMM or the subject having an increased likelihood of stable SMM is prognosed or diagnosed as having an increased likelihood of high-risk SMM or an increased likelihood of stable SMM according to the method
assaying a plurality of plasma cells using three-dimensional (3D) quantitative fluorescence in situ hybridization (q-FISH) and obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution, the plurality of plasma cells previously obtained from a test sample from a subject having smoldering multiple myeloma (SMM);
applying a classification model to the 3D telomere organization sample signature to obtain an output classification of stable SMM or high-risk SMM, the classification model consisting of the telomere parameters: a) nuclear telomere distribution, a/c ratio, and total telomere length; b) a/c ratio and telomere numbers; c) a/c ratio and nuclear telomere distribution, or d) a/c ratio and telomere aqggegates; and
providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years.

20. (canceled)

21. (canceled)

22. (canceled)

23. An assay for selecting therapy for a subject having smoldering multiple myeloma (SMM), the assay comprising subjecting a sample comprising a plurality of plasma cells from the subject to three-dimensional (3D) quantitative fluorescence in situ hybridization (q-FISH); obtaining a 3D telomere organization sample signature, the 3D telomere organization sample signature comprising at least one telomere parameter selected from total telomere length, average telomere length, telomere numbers, telomere aggregates, a/c ratio, nuclear volume, and nuclear telomere distribution; applying a classification model to the 3D telomere organization sample signature to obtain an output classification of stable SMM or high-risk SMM, the model being a model comprising one or more of the at least one telomere parameter; providing the clinical outcome prognosis or the diagnosis to the subject according to the output classification, the clinical outcome prognosis or the diagnosis being an increased likelihood of stable SMM or an increased likelihood of high-risk SMM, wherein the subject with increased likelihood of high-risk SMM is likely to progress to multiple myeloma (MM) within 2 years and the subject with increased likelihood of stable SMM is not likely to progress to MM within 5 years; and selecting a therapy according to claim 12 for the subject when the subject is identified as having an increased likelihood of high-risk SMM, or monitoring the subject when the subject is identified as having an increased likelihood of stable SMM.
